(19) United States
(12) Patent Application Publication
(10) Pub. No.: US 2025/0340836 A1
(43) Pub. Date: Nov. 6, 2025

(54) METHODS FOR PRODUCTION OF IPSCS

(71) Applicant: New York Stem Cell Foundation, Inc., New York, NY (US)

(72) Inventor: Howard Kim, Long Island City, NY (US)

(21) Appl. No.: 18/854,203

(22) PCT Filed: Apr. 7, 2023
(86) PCT No.: PCT/US2023/017855

§ 371 Date: Oct. 4, 2024

Related U.S. Application Data
(60) Provisional application No. 63/329,097, filed on Apr. 8, 2022.

(51) Int. Cl. C12N 5/079; A61K 35/30; C12N 5/00; C12N 5/074; C12N 15/86; C12Q 1/686

(52) U.S. Cl. CPC C12N 5/0621; A61K 35/30; C12N 5/0696; C12N 5/562; C12N 15/86; C12Q 1/686; C12N 2500/98; C12N 2501/02; C12N 2501/105; C12N 2501/999; C12N 2506/45; C12N 2509/00; C12N 2513/00; C12N 2533/52; C12N 2760/18043

(57) ABSTRACT

Provided herein are methods of producing and manufacturing iPSCs, such as iPSCs essentially free of exogeneous viral residuals, and RPE cells. Such methods may comprise cell seeding at low density (e.g., single-cell passaging) and/or culturing at elevated temperature.

### CROSS-REFERENCE TO RELATED APPLICATIONS

This application claims the benefit of and priority to U.S. Provisional Patent Application No. 63/329,097, filed Apr. 8, 2022, the disclosure of which is hereby incorporated by reference in its entirety for all purposes.

### BACKGROUND

Induced pluripotent stem cells (iPSCs) reprogrammed using non-integrating viral or non-viral based reprogramming system typically carry residual copies of the vectors following initial reprogramming. For clinical application, it is important that these exogenous vectors are cleared in the iPSCs, to reduce the high number of passages or subcloning procedures. Thus, there remains the need for an efficient process to produce iPSCs free of exogeneous materials.

### SUMMARY

The invention is based, in part, upon the development of new modified process for iPSC production and manufacturing.

Accordingly, in one aspect, the present disclosure provides a method of producing a clonal population of induced pluripotent stem cells (iPSCs) that is essentially free of exogeneous vector residuals, the method comprising, in the following order:
a) obtaining a starting population of iPSCs produced using a reprogramming vector;
b) seeding the iPSCs at a low density and culturing in a culture medium;
c) optionally repeating step b) for one or more times;
d) optionally culturing the iPSCs at an elevated temperature;
e) selecting a single iPSC colony and passaging for 1-8 times to produce the population of iPSCs that is essentially free of exogeneous vector residuals.

In another aspect, the present disclosure provides a method of manufacturing induced pluripotent stem cells (iPSCs) essentially free of exogeneous vector residuals, comprising, in the following order:
a) obtaining a starting population of iPSCs produced using a reprogramming vector;
b) seeding the iPSCs at a low density and culturing in a culture medium;
c) optionally repeating step b) for one or more times;
d) optionally culturing the iPSCs at an elevated temperature;
e) selecting a single iPSC colony and passaging for 1-8 times to yield the iPSCs essentially free of exogeneous vector residuals.

In some embodiments, the starting population of iPSCs is produced from somatic cells of a human individual. In some embodiments, the somatic cells are blood cells. In some embodiments, the blood cells are peripheral blood cells. In some embodiments, the blood cells are CD34+ or CD71+ enriched cells.

In some embodiments, the starting population of iPSCs is a polyclonal pool of iPSCs. In some embodiments, step a) comprises i) generating a polyclonal pool of iPSCs produced using a reprogramming vector; ii) optionally passaging the iPSCs one or more times; and iii) optionally cryopreserving iPSCs resulting from step i) or step ii), thereby giving the starting population of iPSCs.

In some embodiments, starting population of iPSCs has not been passaged or has been passaged once prior to its use in the method.

In some embodiments, the reprograming vector is a non-integrating viral vector. In some embodiments, the viral vector is a Sendai viral vector, wherein the Sendai viral vector comprises one or more temperature-sensitive mutations.

In some embodiments, step b) is repeated for one time. In some embodiments, step b) is repeated for two times.

In some embodiments, step b), each time performed, independently comprises dissociating the iPSCs into essentially single cells prior to seeding. In some embodiments, step b), each time performed, independently comprises seeding the iPSCs at a density of about 1 to about 1500 cells/cm², such as about 140 to about 350 cells/cm² or about 470 to about 1150 cells/cm².

In some embodiments, step b), when performed the last time, comprises seeding the iPSCs at a clonal density. In some embodiments, the clonal density is about 140 to about 350 cells/cm², such as about 340 cells/cm².

In some embodiments, step b), each time performed, independently comprises seeding the iPSCs in the culture medium supplemented with a Rho-associated protein kinase (ROCK) inhibitor. In some embodiments, the culture medium of step b) is a fully defined medium. In some embodiments, the culture medium is Essential 8 medium.

In some embodiments, step b), each time performed, independently comprises culturing the iPSCs at about 37.0° C. to about 39.0° C.

In some embodiments, step b), each time performed, independently comprises seeding the iPSCs at a density of about 140 to about 350 cells/cm² or about 470 to about 1150 cells/cm² in the culture medium supplemented with a ROCK inhibitor and subsequently culturing in the culture medium at about 37.0° C. in 5% CO₂ incubation atmosphere until single iPSC colonies emerge.

In some embodiments, step b), when performed the last time, comprises seeding the iPSCs at a density of about 140 to about 350 cells/cm² in the culture medium supplemented with a ROCK Inhibitor and subsequently culturing the iPSCs in the culture medium at about 37.0° C. in 5% CO₂ incubation atmosphere for 1-3 days.

In some embodiments, step d) is not performed. In some embodiments, step d) is performed. In some embodiments, step d) comprises culturing the iPSCs in Essential 8 medium in 5% CO₂ incubation atmosphere. In some embodiments, the elevated temperature is about 38.0° C. to about 39.0° C. In some embodiments, step d) comprises culturing the iPSCs at the elevated temperature for 5-8 days. In some embodiments, step d) further comprises culturing the iPSCs at about 37.0° C. for at least 1 day.

In some embodiments, step d) comprises culturing the iPSCs in Essential 8 medium at about 38.0° C. to about 39.0° C. in 5% CO₂ incubation atmosphere for about 6 days and subsequently at about 37.0° C. in 5% CO₂ incubation atmosphere for 1-2 days.

In some embodiments, the essentially free of exogeneous viral residuals is determined by quantitative real-time polymerase chain reaction (qRT-PCR) or quantitative polymerase chain reaction (qPCR).

In another aspect, provided herein is a method of producing a clonal population of induced pluripotent stem cells (iPSCs) that is essentially free of exogeneous viral residuals, the method comprising, in the following order:
a) obtaining a starting population of iPSCs produced using a Sendai viral vector containing temperature-sensitive mutations,
b1) dissociating the iPSCs into essentially single cells and seeding at a density of about 470 to about 1150 cells/cm² in a culture medium supplemented with a ROCK inhibitor and subsequently culturing in the culture medium at about 37.0° C. in 5% CO₂ incubation atmosphere until single iPSC colonies emerge;
b2) dissociating the iPSCs into essentially single cells and seeding at a density of about 140 to about 350 cells/cm² in the culture medium supplemented with a ROCK inhibitor and subsequently culturing in the culture medium at about 37° C. in 5% CO₂ incubation atmosphere for about 3 days;
c) culturing the iPSCs in the culture medium at about 38.0° C. to about 39.0° C. in 5% CO₂ incubation atmosphere for about 6 days and subsequently at about 37.0° C. in 5% CO₂ incubation atmosphere for 1-2 days; and
d) selecting a single iPSC colony and culturing for less than 8 passages to produce the population of iPSCs that is essentially free of exogeneous viral residuals.

In another aspect, provided herein is a method of manufacturing induced pluripotent stem cells (iPSCs) essentially free of exogeneous viral residuals, comprising, in the following order:
a) obtaining a starting population of iPSCs produced using a Sendai viral vector containing temperature-sensitive mutations,
b1) dissociating the iPSCs into essentially single cells and seeding at a density of about 470 to about 1150 cells/cm² in a culture medium supplemented with a ROCK inhibitor and subsequently culturing in the culture medium at about 37.0° C. in 5% CO₂ incubation atmosphere until single iPSC colonies emerge;
b2) dissociating the iPSCs into essentially single cells and seeding at a density of about 140 to about 350 cells/cm² in the culture medium supplemented with a ROCK inhibitor and subsequently culturing in the culture medium at about 37.0° C. in 5% CO₂ incubation atmosphere for about 3 days;
c) culturing the iPSCs at in the culture medium at about 38.0° C. to about 39.0° C. in 5% CO₂ incubation atmosphere for about 6 days and subsequently at about 37.0° C. in 5% CO₂ incubation atmosphere for 1-2 days; and
d) selecting a single iPSC colony and culturing for less than 8 passages to yield iPSCs essentially free of exogeneous viral residuals.

In another aspect, the present application provides a method of producing human retinal pigment epithelial (RPE) cells, comprising, in the following order:
a) obtaining a population of iPSCs that is essentially free of exogeneous viral residuals produced according to any one of claims 1 and 3-32;
b) seeding the iPSCs and culturing in a retinal induction medium to initiate differentiation of the cells into retinal lineage cells;
c) culturing the retinal lineage cells in a retinal differentiation medium to further differentiate the retinal lineage cells;
d) culturing the cells in retinal medium to form differentiating RPE cells; and
e) culturing the differentiating RPE cells in a RPE maturation medium, thereby producing human RPE cells.

In another aspect, the present application provides a method of manufacturing human retinal pigment epithelial (RPE) cells, comprising:
a) obtaining a population of iPSCs essentially free of exogeneous viral residuals according to any one of claims 1-33;
b) seeding the iPSCs and culturing in a retinal induction medium to initiate differentiation of the cells into retinal lineage cells;
c) culturing the retinal lineage cells in a retinal differentiation medium to further differentiate the retinal lineage cells;
d) culturing the cells in retinal medium to form differentiating RPE cells; and
e) culturing the differentiating RPE cells in a RPE maturation medium, thereby yielding human RPE cells.

In some embodiments, the provided method does not comprise the formation of embryoid bodies.

In some embodiments, the iPSCs of step a) have been dissociated into single cells.

In some embodiments, step b) comprises dissociating the iPSCs into essentially single cells prior to seeding. In some embodiments, step b) comprises seeding the iPSCs: i) at a cell density of about 5000 to about 40000 cells/cm2; ii) without a feeder layer; iii) in a fully defined culture medium; and/or iv) in a xeno-free culture medium. In some embodiments, b) comprises culturing iPSCs on a matrix. In some embodiments, the matrix comprises at least one recombinant cellular adhesion protein, such as laminin, vitronectin, or fibronectin. In some embodiments, the cellular adhesion protein is human protein.

In some embodiments, the retinal induction medium comprises a WNT pathway inhibitor, a TGFβ pathway inhibitor, a BMP pathway inhibitor, and insulin growth factor 1 (IGF1). In some embodiments, the retinal differentiation medium comprises a WNT pathway inhibitor, a TGFβ pathway inhibitor, a BMP pathway inhibitor, a MEK inhibitor, and IGF1.

In some embodiments, step e) comprises dissociating the differentiating RPE cells, reseeding the RPE cells, and culturing the RPE cells in the RPE maturation medium, wherein the RPE maturation medium comprises a MEK inhibitor. In some embodiments, the RPE cells are reseeded on a degradable scaffold in the RPE maturation medium. In some embodiments, the RPE maturation medium comprises at least one primary cilium inducer, such as prostaglandin E2 (PGE2) or aphidicolin.

In some embodiments, the provided method further comprises cryopreserving the human RPE cells.

In another aspect, provided herein is a method for producing human retinal pigment epithelial (RPE) cells, comprising, in the following order:
a) obtaining a population of iPSCs that is essentially free of exogeneous viral residuals produced according to any one of claims 1 and 3-32, and dissociating the iPSCs into essentially single cells in a fully defined medium;
b) seeding the iPSCs and culturing on laminin, vitronectin, or a combination thereof, in a retinal induction medium comprising LDN193189, CKI-7, and SB431542 to initiate differentiation of the cells into retinal lineage cells;
c) culturing the retinal lineage cells in a retinal differentiation medium comprising LDN193189, CKI-7, SB431542, and PD0325901 to further differentiate the retinal lineage cells;
d) culturing the cells in retinal medium comprising nicotinamide and Activin A to form differentiating RPE cells; and
e) culturing the differentiating RPE cells in a RPE maturation medium, thereby producing human RPE cells;
wherein the method does not comprise the formation of embryoid bodies.

In another aspect, provided herein is a method for manufacturing human retinal pigment epithelial (RPE) cells, comprising:
a) obtaining iPSCs essentially free of exogeneous viral residuals according to any one of claims 1-33, and dissociating the iPSCs into essentially single cells in a fully defined medium;
b) seeding the iPSCs and culturing on laminin, vitronectin, or a combination thereof, in a retinal induction medium comprising LDN193189, CKI-7, and SB431542 to initiate differentiation of the cells into retinal lineage cells;
c) culturing the retinal lineage cells in a retinal differentiation medium comprising LDN193189, CKI-7, SB431542, and PD0325901 to further differentiate the retinal lineage cells;
d) culturing the cells in retinal medium comprising nicotinamide and Activin A to form differentiating RPE cells; and
e) culturing the differentiating RPE cells in a RPE maturation medium, thereby yielding human RPE cells;
wherein the method does not comprise the formation of embryoid bodies.

In another aspect, provided herein is a pharmaceutical composition comprising human RPE cells produced or manufactured according to a method described herein, a pharmaceutically acceptable carrier, and optionally a scaffold. In some embodiments, the scaffold is a poly(lactic-co-glycolic acid) (PLGA) scaffold.

### BRIEF DESCRIPTIONS OF THE DRAWINGS

FIGS. 1A, 1B, and 1C are phase-contrast microscopic images of early-passage colonies of iPSC reprogrammed with CytoTune Sendai virus vectors at 37° C. (FIG. 1A) or 38.5° C. (FIG. 1B and FIG. 1C). When cultured in the latter condition, a subset of colonies shows morphological signs of differentiation, which may serve as a selection event for clones that may be dependent on the viral vector for maintaining pluripotency.

FIGS. 2A and 2B show iPSCs cultured constantly at 37° C. (FIG. 2A) or with elevated temperature at 38.5° C. (FIG. 2B) stained with an anti-Sendai antibody. Sendai viral vectors were present in some iPSC colonies when cultured at 37° C., but cleared from those treated with elevated temperature. An iPSC line that has been confirmed to be clear for Sendai Virus via RT-qPCR was used as the control.

FIG. 3A is the raw data plot showing signals of Sendai virus during the PCR amplification. FIG. 3B depicts Sendai virus copies for clones from iPSCs cultured with elevated temperature at Passage 2, and measured at both Passages 4 and 5, demonstrating Sendai virus clearance to all clones.

FIG. 4 depicts Sendai virus copies for different clones from iPSCs cultured with a method described in Example 3 and shows Sendai virus clearance to all clones.

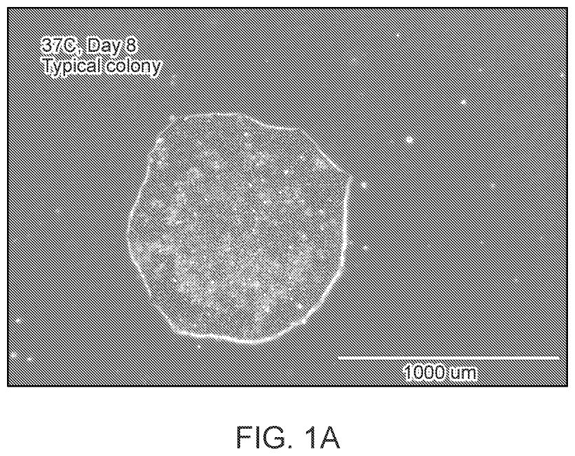

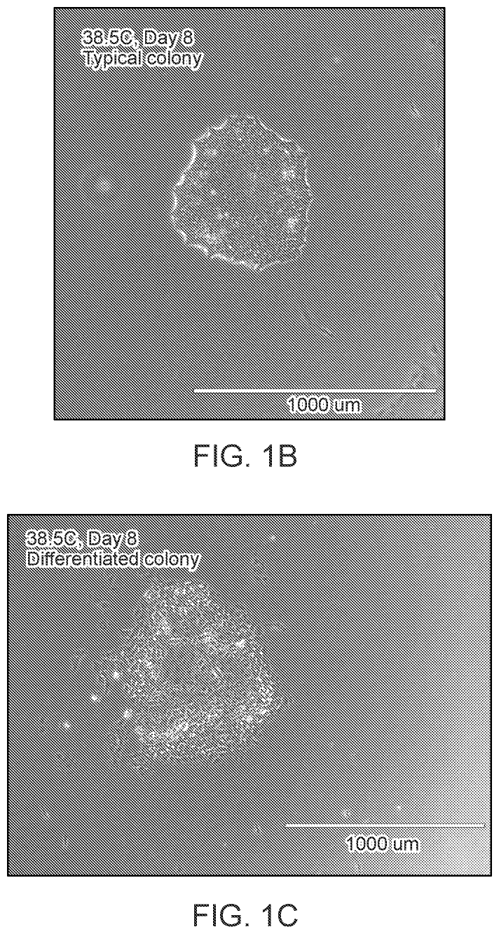

### DETAILED DESCRIPTION

### Definitions

As used herein, the singular forms “a,” “an,” and “the” include plural referents, unless the context clearly dictates otherwise.

As used herein, “about” is understood as within a range of normal tolerance in the art, for example within 2 standard deviations of the mean. “About” can be understood as within 10%, such as 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, or 0.01%, of the value. Unless otherwise clear from the context, all numerical values provided herein are modified by the term “about.”

An “allele” refers to one of two or more forms of a gene. Diploid organisms such as humans contain two copies of each chromosome, and thus carry one allele on each. The term “homozygous” means containing two of the same alleles at a particular locus; the term “heterozygous” means containing two different alleles at a particular locus.

As used herein, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

As used herein, “cell” refers to a structural and functional unit of an organism that can replicate independently, is enclosed by a membrane, and contains biomolecules and genetic material. Cells used herein may be naturally-occurring cells or artificially modified cells (e.g., fusion cells or genetically modified cells).

As used herein, “cell population” or “population of cells” refers to a plurality of, or a group of, cells, typically of a common type. The cell population can be derived from a common progenitor or may comprise more than one cell type. A “clonal” cell population refers to a cell population from a single cell, such that all cells in said population arise from the origin single cell. In some embodiments, a clonal population of cells may be obtained by plating the starting cells at a “clonal density,” which refers to a density at which the starting cells are sparsely plated such that each essentially divides individually during expansion and the resulting colonies are essentially not in contact with one another. An “enriched” cell population refers to a cell population derived from a starting cell population (e.g., an unfractionated, heterogeneous cell population) that contains a greater percentage of a specific cell type than the percentage of that cell type in the starting population. The cell populations may be enriched for one or more cell types and/or depleted of one or more cell types.

Wherever embodiments are described with the language “comprising,” otherwise analogous embodiments described in terms of “consisting of” and/or “consisting essentially of” are included.

As used herein, the term “defined” or “fully defined,” when used in relation to a medium, an extracellular matrix, or a culture condition, refers to a medium, an extracellular matrix, or a culture condition in which the chemical composition and amounts of essentially all the components are known. For example, a defined medium does not contain undefined factors such as in fetal bovine serum, bovine serum albumin or human serum albumin. Generally, a defined medium comprises a basal media (e.g., Dulbecco's Modified Eagle's Medium (DMEM), F12, or Roswell Park Memorial Institute Medium (RPMI) 1640, containing amino acids, vitamins, inorganic salts, buffers, antioxidants, and energy sources), which is supplemented with recombinant albumin, chemically defined lipids, and recombinant insulin. An exemplary fully defined medium is Essential 8™ medium.

As used herein, “differentiation,” “differentiate,” or “differentiating” refers to the process by which an unspecialized cell becomes a more specialized type with changes in structural and/or functional properties. In some embodiments, “differentiation” refers to the process of a human stem cell acquiring the cell type of a retinal pigment epithelial (RPE) cell with features indicative that said RPE cell is a mature, terminally differentiated cell. The term “differentiated cell” encompasses any somatic cell that is not, in its native form, pluripotent, as that term is defined herein. Thus, the term “differentiated cell” also encompasses cells that are partially differentiated, such as multipotent cells, or cells that are stable, non-pluripotent partially reprogrammed, or partially differentiated cells, generated using any of the compositions and methods described herein. In some embodiments, a differentiated cell is a cell that is a stable intermediate cell, such as a non-pluripotent, partially reprogrammed cell. In some embodiments, the term “differentiated cell” also refers to a cell of a more specialized cell type (e.g., decreased developmental potential) derived from a cell of a less specialized cell type (e.g., increased developmental potential) (e.g., from an undifferentiated cell or a reprogrammed cell) where the cell has undergone a cellular differentiation process. The term “terminally differentiated cell” or “mature cell” refers to a cell that does not undergo further differentiation in its native state without treatment or external manipulation. In some embodiment, a terminally differentiated cell has lost the ability to differentiate into a more specialized cell type. The mature cell typically has altered cellular structure and tissue-specific proteins and is committed to specialized functions. The term “undifferentiated cell” refers to a cell other than terminally differentiated cell. Thus, an undifferentiated cell displays characteristic markers and morphological characteristics that clearly distinguish them from terminally differentiated cells of embryo or adult origin.

The term “embryo” refers to a cellular mass obtained by one or more divisions of a zygote or an activated oocyte with an artificially reprogrammed nucleus.

The term “embryoid bodies” or “EBs” refer to aggregates of pluripotent stem cells that can undergo differentiation into cells of the endoderm, mesoderm, and ectoderm germ layers. The spheroid structures form when pluripotent stem cells aggregate and enable the non-adherent culture of EBs in suspension.

The term “embryonic stem (ES) cell” refers to an undifferentiated pluripotent cell which is obtained from an embryo in an early stage, such as the inner cell mass at the blastocyst stage, or produced by artificial means (e.g., nuclear transfer) and can give rise to any differentiated cell type in an embryo or an adult, including germ cells (e.g., sperm and eggs).

As used herein, “essentially” means almost entirely or completely with respect to a given value, dimension, shape, element, material, or another aspect it modifies. For example, in some embodiments, when used in connection with a value, “essentially” refers to, e.g., at least 90%, 91%, 92%, 93%, 94%, 95%, 96%, 97%, 98%, 99%, or more of the value. In some embodiments, methods provided herein involve dissociating the iPSCs into essentially single cells, which means the iPSCs are almost entirely or completely dissociated into single cells, e.g., with at least 90%, 91%, 92%, 93%, 94%, 95%, 96%, 97%, 98%, 99%, or more of the iPSCs dissociated into single cells.

As used herein, “episomal vector” or “episome” refers to an extrachromosomal DNA molecule that can autonomously replicates and maintains itself in the cytoplasm of a cell. In some embodiments, an episomal vector does not integrated into the genome of the host cell. In some embodiments, an episomal vector is of viral origin. In some embodiments, an episomal vector is of nonviral origin.

As used herein, “essentially free,” when used in connection with a given element or material in a composition, means that the given element or material is not purposefully formulated into the composition, not desired in the composition, not detectable in the composition, and/or present only as a contaminant or in trace amounts. In some embodiments, the composition is essentially free of the given element or material, wherein the given element or material is below 0.05%, preferably below 0.01%, by weight of the compositions. In some embodiments, the composition is essentially free of the given element or material, wherein the given element or material is not detectable using a standard analytical method for said element or material.

The terms “exogenous” or “heterologous,” when used to refer to nucleic acids such as DNA, each refer to nucleic acids that originate from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. In some embodiments, exogenous nucleic acids may express to yield exogenous polypeptides. A “homologous” nucleic acid sequence is a nucleic acid sequence that is naturally associated with a host cell into which it is introduced.

As used herein, “expanding” or “expansion” refers to culturing one or more cells for the purpose of obtaining a larger number of cells in the culture.

As used herein, “feeder layers” or “feeder cells” refer to a coating layer of cells, such as on the bottom of a culture dish. The feeder cells can release nutrients into the culture medium and provide a surface to which other cells, such as pluripotent stem cells, can attach.

As used herein, “feeder-free” or “feeder-independent” refers to a culture supplemented with cytokines and growth factors (e.g., TGFβ, bFGF, or LIF) as a replacement for the feeder cell layer. Thus, “feeder-free” or feeder-independent culture systems and media may be used to culture and maintain pluripotent cells in an undifferentiated and proliferative state. In some cases, feeder-free cultures utilize an animal-based matrix (e.g., MATRIGEL™) or are grown on a substrate such as fibronectin, collagen or vitronectin. These approaches allow human stem cells to remain in an essentially undifferentiated state without the need for mouse fibroblast feeder layers.

As used herein, “haplotype” refers to a combination of alleles at multiple loci along a single chromosome. A haplotype can be based upon a set of single-nucleotide polymorphisms (SNPs) on a single chromosome and/or the alleles in the major histocompatibility complex (MHC). The haplotype of a subject can be readily determined using assays well known in the art.

As used herein, the term “haplotype-matched” is defined as the cell (e.g., iPSC cell) and the subject being treated with the cell or cell derivative share one or more MHC locus haplotypes. The haplotype-matched iPSC cell can be autologous or allogeneic. Autologous cells grown in tissue culture and differentiated, e.g., to RPE cells, are haplotype-matched to the subject.

The term “induced pluripotent stem cells” or “iPSCs” refer to cells generated by reprogramming a somatic cell by expressing or inducing expression of a combination of factors (herein referred to as “reprogramming factors”). iPSCs can be generated using fetal, postnatal, newborn, juvenile, or adult somatic cells. In certain embodiments, factors that can be used to reprogram somatic cells to pluripotent stem cells include, for example, Oct4 (sometimes referred to as Oct 3/4), Sox2, c-Myc, and Klf4, Nanog, and Lin28. In some embodiments, somatic cells are reprogrammed by expressing at least two reprogramming factors, at least three reprogramming factors, or four reprogramming factors to reprogram a somatic cell to a pluripotent stem cell.

As used herein, an “isolated” cell has been substantially separated or purified from other cells in an organism or culture. Isolated cells can be, for example, at least 99%, at least 98% pure, at least 95% pure or at least 90% pure.

As used herein, the term “modified,” when used in reference to a cell, e.g., a mammalian cell, refers to a non-naturally occurring cell in which one or both of the following is manipulated by the hand of man: (1) the cell's genetic content; or (2) expression of one or more genes (e.g., manipulated by overexpression, reduction, and/or abrogation).

As used herein, “pluripotent” refers to the property of a cell to differentiate into all other cell types in an organism, except for extraembryonic, or placental, cells. Pluripotent stem cells are capable of differentiating to cell types of all three germ layers (e.g., ectodermal, mesodermal, and endodermal cell types) even after prolonged culture. In some embodiments, a pluripotent stem cell is an embryonic stem cell derived from the inner cell mass of a blastocyst. In some embodiments, a pluripotent stem cell is an induced pluripotent stem cell derived by reprogramming somatic cells.

As used herein, “pre-confluent” refers to a cell culture in which the proportion of the culture surface which is covered by cells is about 60-80%. Usually, pre-confluent refers to a culture in which about 70% of the culture surface is covered by cells.

As used herein, “PSC culture medium” or “PCM” refers to any growth media that for the culture of pluripotent stem cells. In some embodiments, the PCM comprises a Rock inhibitor.

The term “retina” refers to a light-sensitive layer of tissue which lines the inner surface of the eye. The term “retinal pigment epithelium” or “RPE” refers to a monolayer of pigmented cells between the choroid, a layer filled with blood vessels, and the retina.

As used herein, “retinal lineage cells” refer to cells that can give rise or differentiate to retinal pigment epithelium (RPE) cells. In some embodiments, “mature” RPE cells refer to RPE cells which have downregulated expression of immature RPE markers such as Pax6 and upregulated expression of mature RPE markers such as RPE65. RPE cell “maturation” refers herein to the process by which RPE developmental pathways are modulated to generate mature RPE cells. For example, modulation of cilia function can result in RPE cell maturation.

As used herein, “retinal induction medium” or “RIM” refers to a growth media that comprises a WNT pathway inhibitor and a BMP pathway inhibitor and can result in the differentiation of PSCs to retinal lineage cells. In some embodiments, the RIM also comprises a TGFβ pathway inhibitor.

As used herein, “retinal differentiation medium” or “RDM” refers to a medium that comprises a WNT pathway inhibitor, a BMP pathway inhibitor and a MEK inhibitor and differentiates retinal cells. In some embodiments, the RDM also comprises a TGFβ pathway inhibitor.

As used herein, “retinal medium” or “RM” refers to a growth medium for the culture of retinal cells comprising Activin A and Nicotinamide.

As used herein, “RPE-maturation medium” or “RPE-MM” refers to a medium for the maturation of RPE cells comprising taurine and hydrocortisone. In some embodiments, the RPE-MM also comprises triiodothyronine. In some embodiments, the RPE-MM also comprise PD0325901 or PGE2.

As used herein, “stem cell” refers to a cell that, under suitable conditions, is capable of differentiating into a diverse range of specialized cell types, while under other suitable conditions is capable of self-renewing and remaining in an essentially undifferentiated pluripotent state. The term “stem cell” also encompasses a pluripotent cell, multipotent cell, precursor cell, and progenitor cell. Exemplary human stem cells can be obtained from hematopoietic or mesenchymal stem cells obtained from bone marrow tissue, embryonic stem cells obtained from embryonic tissue, or embryonic germ cells obtained from genital tissue of a fetus. Exemplary pluripotent stem cells can also be produced from somatic cells by reprogramming them to a pluripotent state by the expression of certain transcription factors associated with pluripotency; these cells are called “induced pluripotent stem cells” or “iPSCs.”

As used herein, “substantially the same HLA type” indicates that the HLA type of the donor matches with that of a subject to the extent that the cells obtained by inducing differentiation of iPSCs derived from the donor's somatic cells can be engrafted when they are transplanted to the subject.

As used herein, a “super donor” refers to an individual that are homozygous for certain MHC class I and II genes. Homozygous individuals can serve as super donors and their cells, including tissues and other materials comprising their cells, can be transplanted to individuals that are either homozygous or heterozygous for that haplotype. The super donor can be homozygous for the HLA-A, HLA-B, HLA-C, HLA-DR, HLA-DP or HLA-DQ locus/loci alleles, respectively.

“Vector,” “plasmid,” or “recombinant DNA construct” is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the vector can include a nucleic acid to be transcribed operably linked to a promoter. A “reprogramming vector” comprises one or more nucleic acid sequences encoding one or more reprogramming factors, for example, Oct4, Sox2, c-Myc, Klf4, Nanog, and/or Lin28. In some embodiments, a reprogramming vector encodes for at least two reprogramming factors, at least three reprogramming factors, or four reprogramming factors and is capable of reprogramming a somatic cell to a pluripotent stem cell.

As used herein, the term “vector remnants” or “vector residuals” refers to any remaining vectors or vector fragments in a host cell.

As used herein, the terms “virus vector,” “virus-based vector,” and “viral vector” are used interchangeably and describe a genetically modified virus which was manipulated by recombinant DNA technique such that its entry into a host cell results in a specific biological activity, e.g., the expression of one or more transgenes carried by the vector. In some embodiments, the transgene is a reprogramming factor. A viral vector may or may not be replication competent in the target cell, tissue, or organism. In some embodiments, the viral vector does not alter the host genome. In some embodiments, the viral vector is a “non-integrating viral vector,” the genetic material of which essentially does not integrate into the host genome and remains episomal in the cell cytoplasm. Accordingly, the expression, and presence of, the virus is temporary and is not passed to daughter cells via the host cell genome. On the other hand, generally, integrating viral vectors will incorporate a fragment of their genetic material into the host cell genome; such incorporated material is referred to herein as a “viral vector integration remnants” or “viral vector integration remnant residuals.”

The term “Xeno-free” or “XF” when used in relation to a medium, an extracellular matrix, or a culture condition, refers to a medium, an extracellular matrix, or a culture condition which is essentially free from heterogeneous animal-derived components. For culturing human cells, any proteins of a non-human animal, such as mouse, would be xeno components. In certain aspects, the Xeno-free matrix may be essentially free of any non-human animal-derived components, therefore excluding mouse feeder cells or MATRIGEL™. MATRIGEL™ is a solubilized basement membrane preparation extracted from the Engelbreth-Holm-Swarm (EHS) mouse sarcoma, a tumor rich in extracellular matrix proteins to include laminin (a major component), collagen IV, heparan sulfate proteoglycans, and entactin/nidogen.

Producing and Manufacturing Induced Pluripotent Stem Cells (iPSCs)

In various embodiments, provided herein are iPSCs essentially free of exogeneous vector residuals and methods of production and manufacturing thereof. In some embodiments, methods disclosed herein comprise seeding at low density and/or cell culturing at elevated temperature. In some embodiments, methods disclosed herein further comprise single-cell passaging. In some embodiments, methods disclosed herein enables obtaining iPSCs essentially free of exogeneous vector residuals at an early passage.

In various embodiments, the present disclosure provides a method of producing a population of iPSCs that is essentially free of exogeneous vector residuals. In some embodiments, the population of iPSCs is a clonal population. In various embodiments, the present disclosure provides a method of manufacturing iPSCs essentially free of exogeneous vector residuals.

In some embodiments, a provided method comprises, a) obtaining a starting population of iPSCs produced using a non-integrating reprogramming vector(s); b) seeding the iPSCs at a low density and culturing in a PSC culture medium; c) optionally repeat step b) for one or more times; d) optionally culturing the iPSCs at an elevated temperature; and e) selecting a single iPSC colony and passaging for 1-8 times. In some embodiments, the iPSCs essentially free of exogeneous vector residuals are cryopreserved following production.

In some embodiments, the starting population of iPSCs is produced by a method comprising introducing exogenous polynucleotides encoding one or more reprogramming factors into somatic cells using a reprogramming vector. In some embodiments, the starting population of iPSCs is a polyclonal pool of iPSCs. In some embodiments, the starting population of iPSCs has not been passaged prior to its use in the provided method. In some embodiments, the starting population of iPSCs has been passaged once prior to its use in the provided method.

In some embodiments, step a) comprises: i) generating a polyclonal pool of iPSCs produced using a reprogrammed vector; ii) optionally passaging the iPSCs one or more times; and iii) optionally cryopreserving iPSCs resulting from step i) or step ii), thereby giving the starting population of iPSCs.

In some embodiments, step b) is not repeated. In some embodiments, step b) is repeated for one time. In some embodiments, step b) is repeated for two times.

In some embodiments, step b), each time performed, independently comprises dissociating the iPSCs into essentially single cells prior to seeding. In some embodiments, the iPSCs are dissociated from adherent surfaces by incubation with a cell dissociation enzyme, e.g., trypsin or TrypLE™. In some embodiments, the iPSCs are dissociated into a suspension of essentially single cells by pipetting. In some embodiments, the iPSC suspension of essentially single cells is counted before seeding, for example, by a hemocytometer or an automated cell counter, such as VICELL® or TC20. In some embodiments, the cells are be diluted to a cell density of about 10,000 to about 500,000 cells/mL, about 50,000 to about 200,000 cells/mL, or about 75,000 to about 150,000 cells/mL. In some embodiments, the iPSCs is diluted in a fully defined culture medium such as ESSENTIAL 8™ (E8™) Medium. In some embodiments, a ROCK inhibitor is added to the culture medium to increase iPSC survival after dissociated into essentially single cells and while the cells are not adhered to a culture vessel. In some embodiments, in step b), the iPSCs are seeded in the culture medium supplemented with a ROCK inhibitor. In some embodiments, blebbistatin is added to the culture medium to increase iPSC survival after dissociated into essentially single cells and while the cells are not adhered to a culture vessel.

In some embodiments, wherein step b), each time performed, independently comprises seeding the iPSCs in an appropriate culture vessel, such as a tissue culture plate, a flask, a 6-well plate, a 24-well plate, or a 96-well plate. A culture vessel suitable for culturing the cell(s), e.g., iPSC(s), include, but is not limited to: flask, flask for tissue culture, dish, petri dish, dish for tissue culture, multi dish, micro plate, micro-well plate, multi plate, multi-well plate, micro slide, chamber slide, tube, tray, CELLSTACK® Chambers, culture bag, and roller bottle, as long as it is capable of culturing the cells therein. In some embodiments, the cells may be cultured in a volume of at least or about 0.2 mL, 0.5 mL, 1 mL, 2 mL, 5 mL, 10 mL, 20 mL, 30 mL, 40 mL, 50 mL, 100 mL, 150 mL, 200 mL, 250 mL, 300 mL, 350 mL, 400 mL, 450 mL, 500 mL, 550 mL, 600 mL, 800 mL, 1000 mL, 1500 mL, or any range derivable therein, depending on the needs of the culture. In some embodiments, the culture vessel may be a bioreactor, which may refer to any device or system ex vivo that supports a biologically active environment such that cells can be propagated. The bioreactor may have a volume of at least or about 2 L, 4 L, 5 L, 6 L, 8 L, 10 L, 15 L, 20 L, 25 L, 50 L, 75 L, 100 L, 150 L, 200 L, 500 L, 1 m³, 2 m³, 4 m³, 6 m³, 8 m³, 10 m³, 15 m³, or any range derivable therein. In some embodiments, the iPSCs are seeded in a 6-well plate.

In some embodiments, step b), each time performed, independently comprises seeding the iPSCs at low density. In some embodiments, the iPSCs are seeded, e.g., in a 6-well plate, at a density of about 1 to about 1500 cells/cm², such as about 1 to about 150 cells/cm², about 140 to about 350 cells/cm², about 340 to about 480 cells/cm², about 470 to about 1150 cells/cm², or about 1000 to about 1500 cells/cm². In some embodiments, the iPSCs are seeded, e.g., in a 6-well plate, at a density of about 140 to about 350 cells/cm². In some embodiments, the iPSCs are seeded, e.g., in a 6-well plate, at a density of about 470 to about 1150 cells/cm². In some embodiments, the iPSCs are seeded, e.g., in a 6-well plate, at a density of about 50 cells/cm², about 75 cells/cm², about 100 cells/cm², about 125 cells/cm², about 150 cells/cm², about 175 cells/cm², about 200 cells/cm², about 225 cells/cm², about 250 cells/cm², about 275 cells/cm², about 300 cells/cm², about 325 cells/cm², about 350 cells/cm², about 375 cells/cm², about 400 cells/cm², about 500 cells/cm², about 600 cells/cm², about 700 cells/cm², about 800 cells/cm², about 900 cells/cm², about 1000 cells/cm², about 1100 cells/cm², about 1200 cells/cm², about 1300 cells/cm², about 1400 cells/cm², or about 1500 cells/cm². In some embodiments, the iPSCs are seeded e.g., in a 6-well plate, at a density of about 340 cells/cm².

In some embodiments, step b), when performed the last time, comprises seeding the iPSCs at a clonal density. In some embodiments, the iPSCs are seeded in a 6-well plate at a clonal density. In some embodiments, the clonal density is about 140 to about 350 cells/cm². In some embodiments, the clonal density is about 140 cells/cm², about 150 cells/cm², about 160 cells/cm², about 170 cells/cm², about 180 cells/cm², about 190 cells/cm², about 200 cells/cm², about 210 cells/cm², about 220 cells/cm², about 230 cells/cm², about 240 cells/cm², about 250 cells/cm², about 260 cells/cm², about 270 cells/cm², about 280 cells/cm², about 290 cells/cm², about 300 cells/cm², about 310 cells/cm², about 320 cells/cm², about 330 cells/cm², about 340 cells/cm², or about 350 cells/cm². In some embodiments, the clonal density is about 340 cells/cm². In some embodiments, the iPSCs are seeded in a 96-well plate at a clonal density. In some embodiments, the clonal density is about 0.5 to about 5 cells/cm². In some embodiments, the clonal density is about 0.5 cells/cm², about 1 cells/cm², about 1.5 cells/cm², about 2 cells/cm², about 2.5 cells/cm², about 3 cells/cm², about 3.5 cells/cm², about 4 cells/cm², about 4.5 cells/cm², or about 5 cells/cm². In some embodiments, the clonal density is about 1.5 cells/cm².

Cells, e.g., iPSCs, can be cultured with the nutrients necessary to support the growth of each specific population of cells. Generally, the cells are cultured in growth media including a carbon source, a nitrogen source, and a buffer to maintain pH. The medium can also contain fatty acids or lipids, amino acids (such as non-essential amino acids), vitamin(s), growth factors, cytokines, antioxidant substances, pyruvic acid, buffering agents, and inorganic salts. An exemplary growth medium contains a minimal essential media, such as Dulbecco's Modified Eagle's medium (DMEM) or ESSENTIAL 8™ (E8™) medium, supplemented with various nutrients, such as non-essential amino acids and vitamins, to enhance stem cell growth. Examples of minimal essential media include, but are not limited to, Minimal Essential Medium Eagle (MEM) Alpha medium, Dulbecco's modified Eagle medium (DMEM), RPMI-1640 medium, 199 medium, and F12 medium. Additionally, the minimal essential media may be supplemented with additives such as horse, calf or fetal bovine serum. Alternatively, the medium can be serum free.

In other cases, the growth media may contain “knockout serum replacement,” referred to herein as a serum-free formulation optimized to grow and maintain undifferentiated cells, such as stem cell, in culture. In some embodiments, iPSCs described herein are cultured in a fully defined and feeder free media. In some embodiments, iPSCs described herein are cultured in Essential 8 Medium.

In some embodiments, in step b), iPSC dissociated into essentially single cells are cultured in a fully defined culture medium after seeding, such as Essential 8 Medium optionally supplemented with a ROCK inhibitor. Accordingly, in some embodiments, the PSC culture medium of step b) is Essential 8 Medium. In some embodiments, about 40-48 hours after seeding, the medium is aspirated and fresh PSC culture medium, such as Essential 8 Medium, is added to the culture. In some embodiments, the iPSC dissociated into essentially single cells are cultured in PSC culture medium for about 1, 2, 3, 4, 5, 6, or 7 days after seeding. In some embodiments, the iPSCs are cultured to pre-confluency, e.g., such that each cell essentially divides individually during expansion. In some embodiments, the iPSCs are cultured to pre-confluency such that individual colonies are essentially not in contact with one another.

Cells, e.g., iPSCs, are cultured at appropriate temperatures. In some embodiments, iPSCs described herein are cultured at about 37.0° C. to about 39.0° C. In some embodiments, the iPSCs are cultured at about 36.5° C., about 36.6° C., about 36.7° C., about 36.8° C., about 36.9° C., about 37.0° C., about 37.1° C., about 37.2° C., about 37.3° C., about 37.4° C., about 37.5° C., about 37.6° C., about 37.7° C., about 37.8° C., about 37.9° C., about 38.0° C., about 38.1° C., about 38.2° C., about 38.3° C., about 38.4° C., about 38.5° C., about 38.6° C., about 38.7° C., about 38.8° C., about 38.9° C., about 39.0° C., about 39.1° C., about 39.2° C., about 39.3° C., about 39.4° C., or about 39.5° C.

In some embodiments, step b), each time performed, independently comprises culturing the iPSCs at about 37.0° C. to about 39.0° C. In some embodiments, in step b), the iPSCs are cultured at about 37.0° C.

In some embodiments, the elevated temperature of step d) is about 38.0° C. to about 39.0° C. In some embodiments, the elevated temperature of step d) is about 37.8° C., about 37.9° C., about 38.0° C., about 38.1° C., about 38.2° C., about 38.3° C., about 38.4° C., about 38.5° C., about 38.6° C., about 38.7° C., about 38.8° C., about 38.9° C., about 39.0° C., about 39.1° C., or about 39.2° C. In some embodiments, step d) comprises culturing the iPSCs at the evaluated temperature for about 5, 6, 7, or 8 days, e.g., in a PSC culture medium such as Essential 8 Medium. In some embodiments, step d) comprises culturing the iPSCs at the evaluated temperature for about 6 days.

In some embodiments, step d) further comprises, after culturing the iPSCs at an elevated temperature, culturing the iPSCs at about 37.0° C. for at least 1 day, e.g., for 1 day, 2 day, or 3 days.

Other culturing conditions can further be appropriately defined. In some embodiments, for example, the CO₂ concentration is about 1-10%, for example, about 2-5%, or any range derivable therein; the 02 concentration can be at least, up to, or about 1, 2, 3, 4, 5, 6, 7, 8, 9, 10, 20%, or any range derivable therein.

In some embodiments, the iPSC colony is hand-picked in step e). In some embodiments, the hand-picked iPSC colony passaged for 1, 2, 3, 4, 5, 6, 7, or 8 times.

Any methods known in the art may be used to determine and/or confirm the iPSCs are essentially free of exogeneous vector residuals. In some embodiments, the essentially free of exogeneous vector residuals is determined by a polymerase chain reaction (PCR), e.g., quantitative PCR (qPCR), real-time PCR (RT-PCR), or quantitative real-time RT-PCR (aRT-PCR). In some embodiments, the essentially free of exogeneous vector residuals may be determined by immunostaining.

### Somatic Cell Source

In some embodiments, any cell except for germ cells can be used as starting cells for reprogramming to produce iPSCs. For example, in some embodiments, keratinocytes, fibroblasts, hematopoietic cells, mesenchymal cells, liver cells, or stomach cells are used for reprogramming to produce iPSCs. In some embodiments, T cells may be used as a source of somatic cells for reprogramming. There is no limitation on the degree of cell differentiation or the age of an animal from which cells are collected; hence, in some embodiments, undifferentiated progenitor cells (including somatic stem cells) or finally differentiated mature cells can be used as sources of somatic cells for reprogramming to produce iPSCs.

In some embodiments, somatic cells of a human individual are used for reprogramming to produce iPSCs. In some embodiments, the somatic cells are blood cells, such as peripheral blood cells. In some embodiments, the blood cells are CD34+ enriched. In some embodiments, the blood cells are CD71+ enriched. In one embodiment, the somatic cells are retinal pigment epithelial (RPE) cells. The RPE cell may be an adult or a fetal RPE cell.

In some embodiments, iPSCs can be produced from somatic cells of the subject to be treated, or another subject (“donor”) with the same or substantially the same HLA type as that of the subject (“recipient”). In some embodiments, the major HLAs (e.g., the three major loci of HLA-A, HLA-B and HLA-DR) of the donor are identical to the major HLAs of the recipient. In some embodiments, the donor may be a super donor; thus, iPSCs derived from an MHC homozygous super donor may be used to generate, e.g., RPE cells. Accordingly, the iPSCs derived from a super donor may be transplanted in subjects that are either homozygous or heterozygous for that haplotype. For example, the iPSCs can be homozygous at two HLA alleles such as HLA-A and HLA-B. As such, iPSCs produced from super donors can be used in the methods disclosed herein, to produce RPE cells that can potentially “match” many potential recipients.

### Reprogramming and Genetic Modification

Somatic cells can be reprogrammed to produce iPSCs using any methods known to one of skill in the art, such as those disclosed in, e.g., U.S. Publication Nos. 2009/0246875; 2010/0210014; and 2012/0276636; U.S. Pat. Nos. 8,058,065; 8,129,187; 8,278,620; and PCT Publication No. WO 2007/069666. Generally, one or more nuclear reprogramming factors are used to produce pluripotent stem cells from a somatic cell. In some embodiments, the somatic cells are treated with one or more reprogramming substances (e.g., reprogramming factors), or one or more nucleic acids encoding the reprogramming substances (e.g., vectors encoding the reprogramming factors), for producing iPSCs. Methods for introducing one or more reprogramming substances, or nucleic acids encoding these reprogramming substances, are known in the art, and disclosed for example, in U.S. Publication No. 2012/0196360 and U.S. Pat. No. 8,071,369.

Exemplary reprogramming factor combinations are described in, e.g., U.S. Publication No. 2012/0196360, and include: (1) Oct3/4, Klf4, Sox2, and L-Myc (Sox2 can be replaced with Sox1, Sox3, Sox15, Sox17, or Sox18; Klf4 can be replaced with KM, Klf2, or Klf5); (2) Oct3/4, Klf4, Sox2, L-Myc, TERT, and SV40 Large T antigen (SV40LT); (3) Oct3/4, Klf4, Sox2, L-Myc, TERT, and human papilloma virus (HPV)16 E6; (4) Oct3/4, Klf4, Sox2, L-Myc, TERT, and HPV16 E7 (5) Oct3/4, Klf4, Sox2, L-Myc, TERT, HPV16 E6, and HPV16 E7; (6) Oct3/4, Klf4, Sox2, L-Myc, TERT, and Bmil; (7) Oct3/4, Klf4, Sox2, L-Myc, and Lin28; (8) Oct3/4, Klf4, Sox2, L-Myc, Lin28, S and V40LT; (9) Oct3/4, Klf4, Sox2, L-Myc, Lin28, TERT, and SV40LT; (10) Oct3/4, Klf4, Sox2, L-Myc, and SV40LT; (11) Oct3/4, Esrrb, Sox2, and L-Myc (Esrrb is replaceable with Esrrg); (12) Oct3/4, Klf4, and Sox2; (13) Oct3/4, Klf4, Sox2, TERT, and SV40LT; (14) Oct3/4, Klf4, Sox2, TERT, and HP VI 6 E6; (15) Oct3/4, Klf4, Sox2, TERT, and HPV16 E7; (16) Oct3/4, Klf4, Sox2, TERT, HPV16 E6, and HPV16 E7; (17) Oct3/4, Klf4, Sox2, TERT, and Bmil; (18) Oct3/4, Klf4, Sox2, and Lin28 (19) Oct3/4, Klf4, Sox2, Lin28, and SV40LT; (20) Oct3/4, Klf4, Sox2, Lin28, TERT, and SV40LT; (21) Oct3/4, Klf4, Sox2, and SV40LT; or (22) Oct3/4, Esrrb, and Sox2 (Esrrb may be replaced with Esrrg).

In some embodiments, reprogramming factors used in the methods described herein comprise Oct3/4, Klf4, and Sox2. In some embodiments, the reprogramming factors may also comprise L-Myc, c-Myc, Lin28, Lin28b, and/or Nanog. In some embodiments, reprogramming factors such as Klf4, c-Myc, Lin28, or Nanog can increase reprogramming efficiency. In some embodiments, the reprogramming factors comprise Oct4, Nanog, and Sox2. In some embodiments, the reprogramming factors comprise Oct3/4, Klf4, and Myc. In some embodiments, reprogramming factors used in the methods described herein comprise at least three, or at least four, of Klf4, c-Myc, Oct3/4, Sox2, Nanog, and Lin28. In some embodiments, the reprogramming factors comprise Oct3/4, Sox2, c-Myc, and Klf4. In some embodiments, the reprogramming factors comprise Oct3/4, Klf4, Sox2, and Sal4.

In some embodiments, reprogramming of somatic cells may further comprise contacting the cells with one or more signaling regulators, which may be small molecules, inhibitory nucleotides, expression cassettes, or protein factors. Non-limiting examples of such a regulator include a glycogen synthase kinase 3 (GSK-3) inhibitor, a mitogen-activated protein kinase (MEK) inhibitor, a transforming growth factor beta (TGF-0) receptor inhibitor or signaling inhibitor, leukemia inhibitory factor (LIF), a p53 inhibitor, an NF-kappa B inhibitor, or a combination thereof.

In some embodiments, reprogramming factors are introduced to somatic cells using one or more reprogramming vectors encoding said reprogramming factors. Accordingly, in some embodiments, somatic cells are reprogrammed using reprogramming vectors encoding the one or more reprogramming factors to produce iPSCs, e.g., the starting population of iPSCs of a method described herein. In some embodiments, the reprogramming vectors are viral vectors, such as retrovirus vectors, lentivirus vectors, or Sendai virus vectors. In some embodiments, the reprogramming vectors are non-integrating viral vectors. Non-limiting examples of non-integrating viral vectors include adenovirus vector, alphavirus vector, baculovirus vector, Epstein-Barr virus (EBV)-based vector, picornavirus vector, Sendai virus (SeV) vector, and vaccinia virus vector.

In some embodiment, the non-integrating viral vector is a SeV vector. In some embodiment, the SeV vector comprises one or more temperature-sensitive mutations. In some embodiments, the one or more temperature-sensitive mutations are in the polymerase-related genes, e.g., in the phosphoprotein (P) and/or the large protein (L). In some embodiments, the SeV vector weakly expresses at temperatures greater than 37° C., such as at 38° C. or above. In some embodiments, the SeV vector essentially does not express at temperatures greater than 38° C.

In certain embodiments, reprogramming factors are introduced directly into somatic cells by protein transduction.

In some embodiments, the iPSC may be genetically modified. In some embodiments, iPSCs may be modified to express exogenous nucleic acids using a genetic construct (e.g., vector).

In some embodiments, such a genetic construct includes a tyrosinase enhancer operably linked to a promoter and a nucleic acid sequence encoding a marker. The tyrosinase gene is disclosed, for example, in GENBANK® Accession No. 22173, as available on Jan. 1, 2013. This sequence aligns to chromosome 7 of mouse strain C57BL/6 location 5286971-5291691 (invert orientation). In some embodiments, the tyrosinase enhancer is a retinal pigment epithelium (RPE)-specific enhancer, such as D-MITF, DCT, TYRP1, RPE65, VMD2, MERTK, MYRIP, RAB27A, or a 4721 base pair sequence regulatory element sufficient for expression in RPE cells as described in Murisier et al.

In some embodiments, the promoter is any promoter expressed in RPE cells, e.g., the tyrosinase promoter. In some embodiments, the marker may be a protein (e.g., secreted, cell surface, or internal protein), a nucleic acid (e.g., mRNA or enzymatically active nucleic acid molecule), or a polysaccharide. In some embodiments, the marker may be a fluorescence protein (e.g., green fluorescent protein or red fluorescent protein), an enzyme (e.g., horse radish peroxidase, alkaline phosphatase, firefly/Renilla luciferase, or nanoluc), or another protein. In some embodiments, the marker may be a selectable marker (e.g., an antibiotic resistance marker). In some embodiments, the marker may be identified by a biochemical or enzyme assay or biological response that depends on the function of the gene product. Included are determinants of any such cell components that are detectable by antibody, lectin, probe or nucleic acid amplification reaction that are specific for the marker of the cell type of interest.

In some embodiments, the construct may include other genes, such as genes that may influence stem cell to RPE differentiation, or RPE function, or physiology, or pathology. Accordingly, in some embodiments, the construct may include a nucleic acid that encodes one or more of MITF, PAX6, TFEC, OTX2, LHX2, VMD2, CFTR, RPE65, MFRP, CTRP5, CFH, C3, C2B, APOE, APOB, mTOR, FOXO, AMPK, SIRT1-6, HTRP1, ABCA4, TIMP3, VEGFA, CFI, TLR3, TLR4, APP, CD46, BACE1, ELOLV4, ADAM 10, CD55, CD59, and ARMS2.

In some embodiments, the construct can also include other elements, such as a ribosome binding site for translational initiation (internal ribosomal binding sequences) and a transcription/translation terminator. In some embodiments, the iPSCs are transfected with the construct. Suitable constructs for stable transfection include, but are not limited to, retroviral vectors, lentiviral vectors, and Sendai virus.

### Culture and Maintenance

Once derived, iPSCs can be cultured in a medium sufficient to maintain pluripotency. iPSCs may be used with various media and techniques developed to culture pluripotent stem cells (e.g., embryonic stem cells), such as those described in U.S. Pat. No. 7,442,548 and U.S. Publication No. 2003/0211603. In the case of mouse cells, the culture is carried out with the addition of Leukemia Inhibitory Factor (LIF) as a differentiation suppression factor to an ordinary medium. In the case of human cells, it is desirable that basic fibroblast growth factor (bFGF) be added in place of LIF. Other methods for the culture and maintenance of iPSCs, as would be known to one of skill in the art, may also be used.

In certain embodiments, undefined conditions may be used. For example, in some embodiments, pluripotent cells may be cultured on fibroblast feeder cells or a medium that has been exposed to fibroblast feeder cells in order to maintain the stem cells in an undifferentiated state. In some embodiments, the cell is cultured in the co-presence of mouse embryonic fibroblasts treated with radiation or an antibiotic to terminate the cell division, as feeder cells. In other embodiments, pluripotent cells may be cultured and maintained in an essentially undifferentiated state using a defined, feeder-independent culture system, such as a TESR™ medium or E8™ medium. In various embodiments, iPSCs described herein are cultured in E8™ medium.

In some embodiments, iPSCs can be grown under conditions that are known to differentiate human ES cells into specific cell types and express human ES cell markers, such as SSEA-1, SSEA-3, SSEA-4, TRA-1-60, and TRA-1-81.

### Producing and Manufacturing Retinal Pigment Epithelial (RPE) Cells

In various embodiments, provided herein are methods for differentiating iPSCs, e.g., iPSCs essentially free of exogeneous viral residuals produced or manufactured according to a method disclosed herein, into RPE cells.

The cells in the retina that are directly sensitive to light are the photoreceptor cells. Photoreceptors are photosensitive neurons in the outer part of the retina and can be either rods or cones. In the process of phototransduction, the photoreceptor cells convert incident light energy focused by the lens to electric signals which are then sent via the optic nerve to the brain. Vertebrates have two types of photoreceptor cells including cones and rods. Cones are adapted to detect fine detail, central and color vision, and function well in bright light. Rods are responsible for peripheral and dim light vision. Neural signals from the rods and cones undergo processing by other neurons of the retina.

The retinal pigment epithelium acts as a barrier between the bloodstream and the retina and closely interacts with photoreceptors in the maintenance of visual function. The retinal pigment epithelium is composed of a single layer of hexagonal cells that are densely packed with granules of melanin that absorbs light energy that arrives to the retina. The main functions of the specialized RPE cells include: transport of nutrients such as glucose, retinol, and fatty acids from the blood to the photoreceptors; transport of water, metabolic end products, and ions from the subretinal space to the blood; absorption of light and protection against photooxidation; reisomerization of all-trans-retinol into 11-cis-retinal; phagocytosis of shed photoreceptor membranes; and secretion of various essential factors for the structural integrity of the retina.

The retinal pigment epithelium expresses markers such as cellular retinaldehyde-binding protein (CRALBP), RPE65, best vitelliform macular dystrophy gene (VMD2), and pigment epithelium derived factor (PEDF). Malfunction of the retinal pigment epithelium is associated with a number of vision-altering conditions, such as retinal pigment epithelium detachment, dysplasia, atrophy, retinopathy, retinitis pigmentosa, macular dystrophy, or degeneration.

RPE cells can be characterized based upon their pigmentation, epithelial morphology, and apical-basal polarity. Differentiated RPE cells can be visually recognized by their cobblestone morphology and the initial appearance of pigment. In addition, differentiated RPE cells have transepithelial resistance (TER) and trans-epithelial potential (TEP) across the monolayer (TER>100 ohms·cm²; TEP>2 mV), transport fluid and CO₂ from the apical to basal side, and regulate a polarized secretion of cytokines.

RPE cells express several proteins that can serve as markers for detection by the use of methodologies, such as immunocytochemistry, Western blot analysis, flow cytometry, and enzyme-linked immunoassay (ELISA). For example, RPE-specific markers may include: cellular retinaldehyde binding protein (CRALBP), microphthalmia-associated transcription factor (MITF), tyrosinase-related protein 1 (TYRP-1), retinal pigment epithelium-specific 65 kDa protein (RPE65), premelanosome protein (PMEL17), bestrophin 1 (BEST1), and c-mer proto-oncogene tyrosine kinase (MERTK). RPE cells do not express (at any detectable level) the embryonic stem cells markers Oct-4, nanog, or Rex-2. Specifically, expression of these genes is approximately 100-1000 fold lower in RPE cells than in ES cells or iPSC cells, when assessed by quantitative RT-PCR.

RPE cell markers may be detected at the mRNA level, for example, by reverse transcriptase polymerase chain reaction (RT-PCR), Northern blot analysis, or dot-blot hybridization analysis using sequence-specific primers in standard amplification methods using publicly available sequence data (GENBANK®). Expression of tissue-specific markers as detected at the protein or mRNA level is considered positive if the level is at least or about 2-, 3-, 4-, 5-, 6-, 7-, 8-, or 9-fold, and more particularly more than 10-, 20-, 30, 40-, 50-fold or higher above that of a control cell, such as an undifferentiated pluripotent stem cell or other unrelated cell type.

Dysfunction, injury, and loss of RPE cells are factors of many eye diseases and disorders including age-related macular degeneration (AMD), hereditary macular degenerations including Best disease, and retinitis pigmentosa. A potential treatment for such diseases is the transplantation of RPE cells into the retina of those in need of such treatment. It is speculated that the replenishment of RPE cells by their transplantation may delay, halt or reverse degradation, improve retinal function and prevent blindness stemming from such conditions. However, obtaining RPE cells directly from human donors and embryos is a challenge.

Derivation of RPE Cells from Essentially Single Cell PSCs

In various embodiments, methods are provided for producing or manufacturing RPE cells from an essentially single cell suspension of pluripotent stem cells (PSCs), e.g., iPSCs essentially free of exogeneous viral residuals produced or manufactured according to a method disclosed herein. In some embodiments, the method comprise: a) obtaining a population of iPSCs that is essentially free of exogeneous viral residuals; b) seeding the iPSCs and culturing in a retinal induction medium to initiate differentiation of the cells into retinal lineage cells; c) culturing the retinal lineage cells in a retinal differentiation medium to further differentiate the retinal lineage cells; d) culturing the cells in retinal medium to form differentiating RPE cells; and e) culturing the differentiating RPE cells in a RPE maturation medium, thereby producing RPE cells. In some embodiments, the iPSCs of step a) is dissociated into essentially single cells prior to use in step b). In some embodiments, the method does not include the formation of embryoid bodies. In some embodiments, the RPE cells are cryopreserved following production.

In some embodiments, the iPSCs are cultured to pre-confluency to prevent any cell aggregates. In some embodiments, the iPSCs are dissociated by incubation with a cell dissociation enzyme, such as TRYPSIN™ or TRYPLE™. In some other embodiments, the iPSCs can be dissociated into an essentially single cell suspension by pipetting. In some embodiments, Blebbistatin (e.g., about 2.5 μM) can be added to the medium to increase iPSC survival after dissociation into single cells while the cells are not adhered to a culture vessel. In some embodiments, a ROCK inhibitor instead of Blebbistatin may alternatively be used to increase iPSC survival after dissociated into single cells.

In some embodiments, an accurate count of the seeding density can increase RPE differentiation efficiency from the single cell iPSCs. Accordingly, in some embodiments, the essentially single cell suspension of iPSCs is counted before seeding in step b), e.g., using a hemocytometer or an automated cell counter, such as VICELL® or TC20. In some embodiments, the cells may be diluted to a cell density of about 10000 to about 500000 cells/mL, about 50000 to about 200000 cells/mL, or about 75000 to about 150000 cells/mL. In a non-limiting example, the essentially single cell suspension of iPSCs is diluted to a density of about 100000 cells/mL in a fully defined cultured medium such as ESSENTIAL 8™ (E8™) medium.

Once a single cell suspension of iPSCs is obtained at a known cell density, the cells are generally seeded in an appropriate culture vessel, such as a flask or a tissue culture plate (e.g., 6-well, 24-well, or 96-well plate). In some embodiments, the cells are cultured in a volume of at least or about 0.2 mL, about 0.5 mL, about 1 mL, about 2 mL, about 5 mL, about 10 mL, about 20 mL, about 30 mL, about 40 mL, about 50 mL, about 100 mL, about 150 mL, about 200 mL, about 250 mL, about 300 mL, about 350 mL, about 400 mL, about 450 mL, about 500 mL, about 550 mL, about 600 mL, about 800 mL, about 1000 mL, about 1500 mL, or any range derivable therein, depending on the needs of the culture. In certain embodiments, the culture vessel is a bioreactor, such as a bioreactor having a volume of at least or about 2, 4, 5, 6, 8, 10, 15, 20, 25, 50, 75, 100, 150, 200, 500 liters, 1, 2, 4, 6, 8, 10, 15 cubic meters, or any range derivable therein.

In some embodiments, the iPSCs are plated in step b) at a cell density appropriate for efficient differentiation. In some embodiments, the iPSCs are plated at a cell density of about 1000 to about 75000 cells/cm², such as of about 5000 to about 40000 cells/cm². In some embodiments, the iPSCs are seeded in a 6-well plate at a cell density of about 50000 to about 400000 cells per well. In some embodiments, the iPSCs are seeded in a 6-well plate at a cell density of about 100000, about 150000, about 200000, about 250000, about 300000, or about 350000 cells per well, such as about 200000 cells per well.

In some embodiments, the iPSCs (e.g., in step a) and b)) are cultured on a matrix. PSCs, such as iPSCs, are generally cultured on culture plates coated by one or more cellular adhesion proteins to promote cellular adhesion while maintaining cell viability. For example, preferred cellular adhesion proteins include extracellular matrix proteins such as vitronectin, laminin, collagen, and/or fibronectin, which may be used to coat a culturing surface as a means of providing a solid support for pluripotent cell growth. The term “extracellular matrix” is recognized in the art. Its components include one or more of the following proteins: fibronectin, laminin, vitronectin, tenascin, entactin, thrombospondin, elastin, gelatin, collagen, fibrillin, merosin, anchorin, chondronectin, link protein, bone sialoprotein, osteocalcin, osteopontin, epinectin, hyaluronectin, undulin, epiligrin, and kalinin. The extracellular matrix (ECM) proteins may be of natural origin and purified from human or animal tissues or, alternatively, the ECM proteins may be genetically engineered recombinant proteins or synthetic in nature. The ECM proteins may be a whole protein or in the form of peptide fragments, native or engineered. Examples of ECM protein that may be useful in the matrix for cell culture include laminin, collagen I, collagen IV, fibronectin and vitronectin. In some embodiments, the matrix composition includes synthetically generated peptide fragments of fibronectin or recombinant fibronectin. In some embodiments, the matrix composition is xeno-free. For example, in the xeno-free matrix to culture human cells, matrix components of human origin may be used, wherein any non-human animal components may be excluded.

In some embodiments, the iPSCs are cultured on culture plates coated with a matrix. In some embodiments, the matrix comprises at least one recombinant cellular adhesion protein, such as laminin, vitronectin, or fibronectin. In some embodiments, the cellular adhesion proteins are human proteins. In some embodiments, the total protein concentration in the matrix composition may be about 1 ng/mL to about 1 mg/mL. In some embodiments, the total protein concentration in the matrix composition is about 1 g/mL to about 300 g/mL. In some embodiments, the total protein concentration in the matrix composition is about 5 g/mL to about 200 g/mL.

In some embodiments, the iPSCs are cultured without a feeder layer.

In some embodiments, the single cell iPSCs are cultured in a fully-defined culture medium after plating. In some embodiments, about 18-24 hours after seeding, the medium is aspirated and fresh medium, such as E8™ medium, is added to the culture. In some embodiments, the iPSCs are cultured in the fully defined culture medium for about 1, 2, or 3 days after plating. Preferably, the single cells PSCs are cultured in the fully defined culture medium for about 2 days before proceeding with the differentiation process.

In some embodiments, the iPSCs are cultured in a xeno-free culture medium. In some embodiments, the medium may contain or may not contain any alternatives to serum. The alternatives to serum can include materials which appropriately contain albumin (such as lipid-rich albumin, albumin substitutes such as recombinant albumin, plant starch, dextrans and protein hydrolysates), transferrin (or other iron transporters), fatty acids, insulin, collagen precursors, trace elements, 2-mercaptoethanol, 3′-thiolgiycerol, or equivalents thereto. The alternatives to serum can be prepared by the method disclosed in International Publication No. WO 98/30679, for example. Any commercially available materials can also be used, such as KNOCKOUT™ Serum Replacement (KSR), Chemically-defined Lipid concentrated (Gibco), or GLUTAMAX™ (Gibco)

In some embodiments, the retinal induction medium comprises a WNT pathway inhibitor, a BMP pathway inhibitor, a TGFβ pathway inhibitor, and insulin growth factor 1 (IGF1).

In some embodiments, the retinal differentiation medium comprises a WNT pathway inhibitor, a BMP pathway inhibitor, a TGFβ pathway inhibitor, a MEK inhibitor, and IGF1.

Other culturing conditions can be appropriately defined. For example, the culturing temperature can be about 30 to 40° C., for example, at least or about 31, 32, 33, 34, 35, 36, 37, 38, 39° C. In some embodiments, the cells are cultured at 37° C. The CO₂ concentration can be about 1 to 10%, for example, about 2 to 5%, or any range derivable therein. The oxygen tension can be at least, up to, or about 1, 2, 3, 4, 5, 6, 7, 8, 9, 10, 20%, or any range derivable therein.

In some embodiments, the RPE cells are dissociated after culturing in the retinal medium and reseeded on a degradable scaffold in the RPE maturation medium thereby producing mature RPE cells. In some embodiments, the degradable scaffold is a poly(lactic-co-glycolic acid) (PLGA) scaffold.

Derivation of RPE Cells from Embryoid Bodies of PSCs

In some embodiments, RPE cells are derived from iPSCs through a process of differentiation of embryoid bodies (EBs) of the iPSCs into cultures highly enriched for RPE cells. In some embodiments, EBs are produced from iPSCs by the addition of a rho-associated coiled-coil kinase (ROCK) inhibitor and cultured in a first medium comprising two WNT pathway inhibitors and a Nodal pathway inhibitor. The EBs are plated on a MATRIGEL™ coated tissue culture in a second medium that does not comprise basic fibroblast growth factor (bFGF), comprises a Nodal pathway inhibitor, comprises about 20 ng to about 90 ng of Noggin, and comprises about 1 to about 5% knock out serum replacement to form differentiating RPE cells. The differentiating RPE cells are cultured in a third medium comprising ACTIVIN and WNT3a. The RPE cells are then cultured in RPE medium that includes about 5% fetal serum, a canonical WNT inhibitor, a non-canonical WNT inhibitor, and inhibitors of the Sonic Hedgehog and FGF pathways to produce human RPE cells.

### Culturing in Retinal Induction Medium

After the single cell iPSCs have adhered to the culture plate, the cells are cultured in retinal induction medium (RIM) to start the differentiation process into retinal lineage cells. In some embodiments, the media is aspirated each day and replaced with fresh RIM. In some embodiments, the iPSCs are cultured in the RIM for about 1 to about 5 days, such as about 1, 2, 3, 4 or 5 days, such as for about 2 days to produce retinal lineage cells.

In some embodiments, the RIM comprises a WNT pathway inhibitor and can result in the differentiation of PSCs to retinal lineage cells. In some embodiments, the RIM further comprises a TGFβ pathway inhibitor and a BMP pathway inhibitor. One exemplary RIM medium is shown in Table 1. In some embodiments, the RIM can include DMEM and F12 at about a 1:1 ratio.

In some embodiments, the WNT pathway inhibitor is any inhibitor of a member of the WNT family proteins including Wnt1, Wnt2, Wnt2b, Wnt3, Wnt4, Wnt5A, Wnt6, Wnt7A, Wnt7B, Wnt8A, Wnt9A, Wnt10a, Wnt11, and Wnt16. Examples of suitable WNT inhibitors, already known in the art, include, but are not limited to, N-(2-Aminoethyl)-5-chloroisoquinoline-8-sulphonamide dihydrochloride (CKI-7), N-(6-Methyl-2-benzothiazolyl)-2-[(3,4,6,7-tetrahydro-4-oxo-3-phenylthieno[3,2-d]pyrimidin-2-yl)thio]-acetamide (IWP2), N-(6-Methyl-2-benzothiazolyl)-2-[(3,4,6,7-tetrahydro-3-(2-methoxyphenyl)-4-oxothieno[3,2-d]pyrimidin-2-yl)thio]-acetamide (IWP4), 2-Phenoxybenzoic acid-[(5-methyl-2-furanyl)methylene]hydrazide (PNU 74654) 2,4-diamino-quinazoline, quercetin, 3,5,7,8-Tetrahydro-2-[4-(trifluoromethyl)phenyl]-4H-thiopyrano[4,3-d]pyrimidin-4-one (XAV939), 2,5-Dichloro-N-(2-methyl-4-nitrophenyl)benzenesulfonamide (FH 535), N-[4-[2-Ethyl-4-(3-methylphenyl)-5-thiazolyl]-2-pyridinyl]benzamide (TAK 715), Dickkopf-related protein one (DKK1), and Secreted frizzled-related protein (SFRP1) In some embodiments, inhibitors of WNT include antibodies to, dominant negative variants of, and siRNA and antisense nucleic acids that suppress expression of WNT. In some embodiments, inhibition of WNT can also be achieved using RNA-mediated interference (RNAi). In some embodiments, the WNT pathway inhibitor is CKI-7.

In some embodiments, the BMP pathway inhibitor may be an inhibitor of BMP signaling in general or an inhibitor specific for BMP1, BMP2, BMP3, BMP4, BMP5, BMP6, BMP7, BMP8a, BMP8b, BMP10, or BMP15. Exemplary BMP inhibitors include, but are not limited to, 4-(6-(4-(piperazin-1-yl)phenyl)pyrazolo[1,5-a]pyrimidin-3-yl)quinoline hydrochloride (LDN193189), 6-[4-[2-(1-Piperidinyl)ethoxy]phenyl]-3-(4-pyridinyl)-pyrazolo[1,5-a]pyrimidine dihydrochloride (Dorsomorphin), 4-[6-[4-(1-Methylethoxy)phenyl]pyrazolo[1,5-a]pyrimidin-3-yl]-quinoline (DMH1), 4-[6-[4-[2-(4-Morpholinyl)ethoxy]phenyl]pyrazolo[1,5-a]pyrimidin-3-yl]quinoline (DMH-2), and 5-[6-(4-Methoxyphenyl)pyrazolo[1,5-a]pyrimidin-3-yl]quinoline (ML 347). In some embodiments, the BMP pathway inhibitor is LDN193189.

In some embodiments, TGFβ pathway inhibitors may include any inhibitors of TGFβ signaling in general. For example, in some embodiments, the TGFβ pathway inhibitor is 4-[4-(1,3-benzodioxol-5-yl)-5-(2-pyridinyl)-1H-imidazol-2-yl]benzamide (SB431542), 6-[2-(1,1-Dimethylethyl)-5-(6-methyl-2-pyridinyl)-1H-imidazol-4-yl]quinoxaline (SB525334), 2-(5-Benzo[1,3]dioxol-5-yl-2-ieri-butyl-3H-imidazol-4-yl)-6-methylpyridine hydrochloride hydrate (SB-505124), 4-(5-Benzol[1,3]dioxol-5-yl-4-pyridin-2-yl-1H-imidazol-2-yl)-benzamide hydrate, 4-[4-(1,3-Benzodioxol-5-yl)-5-(2-pyridinyl)-1H-imidazol-2-yl]-benzamide hydrate, left-right determination factor (Lefty), 3-(6-Methyl-2-pyridinyl)-N-phenyl-4-(4-quinolinyl)-1H-pyrazole-1-carbothioamide (A 83-01), 4-[4-(2,3-Dihydro-1,4-benzodioxin-6-yl)-5-(2-pyridinyl)-1H-imidazol-2-yl]benzamide (D 4476), 4-[4-[3-(2-Pyridinyl)-1H-pyrazol-4-yl]-2-pyridinyl]-N-(tetrahydro-2H-pyran-4-yl)-benzamide (GW 788388), 4-[3-(2-Pyridinyl)-1H-pyrazol-4-yl]-quinoline (LY 364847), 4-[2-Fluoro-5-[3-(6-methyl-2-pyridinyl)-1H-pyrazol-4-yl]phenyl]-1H-pyrazole-1-ethanol (R 268712), or 2-(3-(6-Methylpyridine-2-yl)-1H-pyrazol-4-yl)-1,5-naphthyridine (RepSox). In some embodiments, the TGFβ pathway inhibitor is SB431542.

In some embodiments, the RIM comprises a WNT pathway inhibitor, such as CKI-7, a BMP pathway inhibitor, such as LDN193189, and a TGFβ pathway inhibitor, such as SB431542. For example, in some embodiments, the RIM comprises about 5 nM to about 50 nM, such as about nM, of LDN193189, about 0.1 μM to about 5 μM, such as about 0.5 μM, of CKI-7, and about 0.5 μM to about 10 μM, such as about 1 μM, of SB431542. In some embodiments, the RIM can include knockout serum replacement, such as about 1% to about 5%, MEM non-essential amino acids (NEAA), sodium pyruvate, N-2 supplement, B-27 supplement, ascorbic acid, and insulin growth factor 1 (IGF1). In some embodiments, the IGF1 is animal free IGF1 (AF-IGF1) and is comprised in the RIM from about 0.1 ng/mL to about 10 ng/mL, such as about 1 ng/mL.

### Culturing in Retinal Differentiation Medium

The retinal lineage cells can then be cultured in retinal differentiation medium (RDM) for further differentiation. In some embodiments, the cells are given fresh RDM each day after aspiration of the media from the previous day. In some embodiments, the cells are cultured in the RDM for about 2, 3, 4, 5, 6, 7, 8, 9, 10, 11, 12, 13, 14, 15, or 16 days, such as for about 7 days to derive differentiated retinal cells.

In some embodiments, the RDM comprises a WNT pathway inhibitor, a BMP pathway inhibitor, a TGFβ pathway inhibitor, and a MEK inhibitor. In some other embodiments, the RDM comprises a WNT pathway inhibitor, a BMP pathway inhibitor, a TGFβ pathway inhibitor, and a bFGF inhibitor.

In some embodiments, the WNT pathway inhibitor is a WNT pathway inhibitor disclosed herein. In some embodiments, the WNT pathway inhibitor is CKI-7.

In some embodiments, the BMP pathway inhibitor is a BMP pathway inhibitor disclosed herein. In some embodiments, the BMP pathway inhibitor is LDN193189.

In some embodiments, the TGFβ pathway inhibitor is a TGFβ pathway inhibitor disclosed herein. In some embodiments, the TGFβ pathway inhibitor is SB431542.

In some embodiments, a MEK inhibitor is any chemical or drug that inhibits the mitogen-activated protein kinase enzymes MEK1 or MEK2. For example, in some embodiments, MEK inhibitors include N-[(2R)-2,3-Dihydroxypropoxy]-3,4-difluoro-2-[(2-fluoro-4-iodophenyl)amino]-benzamide (PD0325901), N-[3-[3-cyclopropyl-5-(2-fluoro-4-iodoanilino)-6,8-dimethyl-2,4,7-trioxopyrido[4,3-d]pyrimidin-1-yl]phenyl]acetamide (GSK1120212), 6-(4-bromo-2-fluoroanilino)-7-fluoro-N-(2-hydroxyethoxy)-3-methylbenzimidazole-5-carboxamide (MEK162), N-[3,4-difluoro-2-(2-fluoro-4-iodoanilino)-6-methoxyphenyl]-1-(2,3-dihydroxypropyl)cyclopropane-1-sulfonamide (RDEA119), and 6-(4-bromo-2-chloroanilino)-7-fluoro-N-(2-hydroxyethoxy)-3-methylbenzimidazole-5-carboxamide (AZD6244). In some embodiments, the MEK inhibitor is PD0325901.

In some embodiments, bFGF inhibitors include, but are not limited to, N-[2-[[4-(Diethylamino)butyl]amino-6-(3,5-dimethoxyphenyl)pyrido[2,3-d]pyrimidin-7-yl]-N′-(1,1-dimethylethyl)urea (PD173074), 2-(2-Amino-3-methoxyphenyl)-4H-1-benzopyran-4-one (PD 98059), 1-tert-Butyl-3-[6-(2,6-dichlorophenyl)-2-[[4-(diethylamino)butyl]amino]pyrido[2,3-d]pyrimidin-7-yl]urea (PD161570), 6-(2,6-Dichlorophenyl)-2-[[4-[2-(diethylamino)ethoxy]phenyl]amino]-8-methyl-pyrido[2,3-d]pyrimidin-7(8H)-one dihydrochloride hydrate (PD166285), N-[2-Amino-6-(3,5-dimethoxyphenyl)pyrido[2,3-d]pyrimidin-7-yl]-N′-(1,1-dimethylethyl)-urea (PD166866), and MK-2206.

In some embodiments, the RDM comprises a WNT pathway inhibitor, such as CKI-7, a BMP pathway inhibitor, such as LDN193189, a TGFβ pathway inhibitor, such as SB431542, and a MEK inhibitor, such as PD0325901. In some embodiments, the RDM comprises LDN193189, CKI-7, SB431542, and PD0325901. In some embodiments, the concentrations of the Wnt pathway inhibitor, BMP pathway inhibitor, and TGFβ pathway inhibitor are higher in the RDM as compared to the RIM, such as about 9 to about 11 times higher, such as about 10 times higher. In some embodiments, the RDM comprises about 50 nM to about 200 nM, such as about 100 nM of LDN193189, about 1 μM to about 10 μM, such as about 5 μM, of CKI-7, about 1 μM to about 50 μM, such as about 10 μM, of SB431542, and about 0.1 μM to about 10 μM, such as about 1 μM, 2 μM, 3 μM, 4 μM, 5 μM, 6 μM, 7 μM, 8 μM, or 9 μM of PD0325901. One exemplary RDM medium is shown in Table 1.

In some embodiments, the RDM comprises DMEM and F12 at about a 1:1 ratio, knockout serum replacement (e.g., about 1% to about 5%, such as about 1.5%), MEM NEAA, sodium pyruvate, N-2 supplement, B-27 supplement, ascorbic acid, and IGF1 (e.g., about 1 ng/mL to about 50 ng/mL, such as about 10 ng/mL).

### Culturing in Retinal Medium

Next, the differentiated retinal cells can be even further differentiated by culturing the cells in retinal medium (RM). In some embodiments, the medium is changed daily with room temperature RM. In some embodiments, the cells are cultured in the RM for about 8, 9, 10, 11, 12, 13, 14, 15, 16 or 17 days, such as for about 10 days to derive differentiating RPE cells.

In some embodiments, the RM comprises activin A and optionally nicotinamide. In some embodiments, the RM comprises about 50 to about 200 ng/mL, such as about 100 ng/mL, of activin A, and about 1 mM to about 50 mM, such as about 10 mM, of nicotinamide. In some other embodiments, the RM can comprise TGF-β pathway activators such as GDF1 and/or WNT pathway activators such as WAY-316606, IQ1, QS11, SB-216763, BIO (6-bromoindirubin-3′-oxime), or 2-amino-4-[3,4-(methylenedioxy)benzyl-amino]-6-(3-methoxyphenyl) pyrimidine. In some other embodiments, the RM can additionally comprise WNT3a. One exemplary RM medium is shown in Table 1.

In some embodiments, the RM can include DMEM and F12 at about a 1:1 ratio, knockout serum replacement at about 1% to about 5%, such as about 1.5%, MEM non-essential amino acids (NEAA), sodium pyruvate, N-2 supplement, B-27 supplement, and ascorbic acid. [0128]

### Culturing in RPE-Maturation Medium

For further differentiation of the RPE cells, the cells may be cultured in RPE maturation medium (RPE-MM). Exemplary RPE-MM media are shown in Table 1. In some embodiments, the RPE-MM comprises about 100 g/mL to about 300 g/mL, such as about 250 g/mL, of taurine, about 10 g/L to about 30 g/L, such as about 20 g/L, of hydrocortisone and about 0.001 g/L to about 0.1 g/L, such as about 0.013 g/L, of triiodothyronine. In some embodiments, the RPE-MM comprises MEM Alpha, N-2 supplement, MEM non-essential amino acids (NEAA), sodium pyruvate, and/or fetal bovine serum (e.g., about 0.5% to about 10%, such as about 1% to about 5%). In some embodiments, the RPE-MM does not include a WNT pathway inhibitor.

In some embodiments, the medium is changed every other day with room temperature RPE-MM. In some embodiments, the cells are cultured in RPE-MM for about 5 to about 10 days, such as about 5 days. Subsequently, in some embodiments, the cells are dissociated, such as with a cell dissociation enzyme, reseeded, and cultured for an additional period of time, such as about to about 30 days, such as about 15 to 20 days, for further differentiation into RPE cells.

RPE cells can be cryopreserved at this stage.

### Maturation of RPE Cells

The RPE cells can then be cultured in the RPE-MM for a continued period of time for maturation. In some embodiments, the RPE cells are grown on culture plates, such as a 6-well, 12-well, 24-well, or 10 cm plate. In some embodiments, the RPE cells can be maintained in RPE-MM for about four to about ten weeks, such as for about six to eight weeks, such as for six, seven, or eight weeks.

In some embodiments of the continued maturation of the RPE cells, the cells are dissociated and reseeded on a degradable scaffold assembly, and cultured for a period of time. In some embodiments, the degradable scaffold assembly is formed by biodegradable polymers, such as polylactic acid (PLA), polyglycolic acid (PGA), polylactic acid-glycolic acid (PLGA), polyorthoesters, polyanhydrides, polyphosphazenes, and combinations thereof. In some embodiments, the degradable scaffold assembly is formed by PLGA.

In some embodiments of the continued maturation of the RPE cells, the cells can be dissociated in a cell dissociated enzyme such as TRYPLE™ and reseeded on a degradable scaffold assembly such as in a specialized SNAPWELL™ design for about one to two weeks in RPE-MM supplemented with a MEK inhibitor (e.g., PD0325901) or a bFGF inhibitor. In some embodiments, methods for culturing RPE cells on a degradable scaffold are described in PCT Publication No. WO 2014/121077, which is incorporated herein by reference in its entirety. Briefly, the main components of this method are a CORNING® COSTAR® SNAPWELL™ plate, abioinert O-ring, and a biodegradable scaffold. SNAPWELL™ plates provide the structure and platform for the biodegradable scaffolds. The microporous membrane that creates an apical and basal side is ideal for providing support to the scaffold as well as isolating the distinct sides of the polarized layer of cells. The ability of the SNAPWELL™ insert to detach the membrane allows the support ring of the insert to be used an anchor for the scaffold. The resulting differentiated, polarized, and confluent monolayers of functional RPE cells can be cryopreserved at this stage (e.g., in xenofree CS10 medium).

In some embodiment, mature RPE cells can be further developed into functional RPE cell monolayers that behave as intact RPE tissue by continued culture in the RPE-MM with additional chemicals or small molecules that promote RPE maturation. Examples of such small molecules include, but are not limited to, primary cilium inducers such as prostaglandin E2 (PGE2) or aphidicolin. In some embodiment, the PGE2 may be added to the RPE-MM at a concentration of about 25 μM to about 250 μM, such as about 50 μM to about 100 μM. In some embodiment, the RPE-MM can comprise canonical WNT pathway inhibitors. Exemplary canonical WNT pathway inhibitors include, but are not limited to, N-(6-Methyl-2-benzothiazolyl)-2-[(3,4,6,7-tetrahydro-4-oxo-3-phenylthieno[3,2-d]pyrimidin-2-yl)thio]-acetamide (IWP2), and 4-(1,3,3a,4,7,7a-Hexahydro-1,3-dioxo-4,7-methano-2H-isoindol-2-yl)-N-8-quinolinyl-Benzamide (endo-IWR1). In some embodiment, the cells are cultured in such medium for an additional period of time, such as an additional about one week to about five weeks, such as about another two to four weeks to obtain mature and functional RPE cell monolayers.

Accordingly, the presently disclosed methods provide mature RPE cells from iPSCs that can be consistently reproduced at a large scale for clinical applications.

### Cryopreservation of RPE Cells

The RPE cells produced by the methods disclosed herein can be cryopreserved, for example, as described in PCT Publication No. 2012/149484. The cells can be cryopreserved with or without a substrate. In some embodiments, the storage temperature ranges from about −50° C. to about −60° C., about −60° C. to about −70° C., about −70° C. to about −80° C., about −80° C. to about −90° C., about −90° C. to about −100° C., and overlapping ranges thereof. In some embodiments, lower temperatures are used for the storage (e.g., maintenance) of the cryopreserved cells. In some embodiments, liquid nitrogen (or other similar liquid coolant) is used to store the cells. In some embodiments, the cells are stored for greater than about 6 hours. In some embodiments, the cells are stored about 72 hours. In some embodiments, the cells are stored 48 hours to about one week. In yet other embodiments, the cells are stored for about 1, 2, 3, 4, 5, 6, 7, or 8 weeks. In some embodiments, the cells are stored for 1, 2, 3, 4, 5, 67, 8, 9, 10, 11 or 12 months. The cells can also be stored for longer times. The cells can be cryopreserved separately or on a substrate, such as any of the substrates disclosed herein.

In some embodiments, additional cryoprotectants can be used. For example, in some embodiments, the cells are cryopreserved in a cryopreservation solution comprising one or more cryoprotectants, such as DM80, serum albumin, such as human or bovine serum albumin. In some embodiments, the solution comprises about 1%, about 1.5%, about 2%, about 2.5%, about 3%, about 4%, about 5%, about 6%, about 7%, about 8%, about 9%, or about 10% dimethylsulfoxide (DMSO). In some embodiments, the solution comprises about 1% to about 3%, about 2% to about 4%, about 3% to about 5%, about 4% to about 6%, about 5% to about 7%, about 6% to about 8%, about 7% to about 9%, or about 8%, to about 10% DMSO or albumin. In a specific embodiment, the solution comprises 2.5% DMSO. In another specific embodiment, the solution comprises 10% DMSO.

In some embodiments, cells may be cooled, for example, at about 1° C./minute during cryopreservation. In some embodiments, the cryopreservation temperature is about −80° C. to about −180° C., or about −125° C. to about −140° C. In some embodiments, the cells are cooled to 4° C. prior to cooling at about 1° C./minute. Cryopreserved cells can be transferred to vapor phase of liquid nitrogen prior to thawing for use. In some embodiments, for example, once the cells have reached about −80° C., they are transferred to a liquid nitrogen storage area. Cryopreservation can also be done using a controlled-rate freezer. Cryopreserved cells may be thawed, e.g., at a temperature of about 25° C. to about 40° C., and typically at a temperature of about 37° C.

### Pharmaceutical Compositions

Also provided herein are pharmaceutical compositions of the RPE cells obtained by the methods disclosed herein. These compositions can include at least about 1×10³ RPE cells, about 1×10⁴ RPE cells, about 1×10⁵ RPE cells, about 1×10⁶ RPE cells, about 1×10⁷ RPE cells, about 1×10⁸ RPE cells, or about 1×10⁹ RPE cells. In certain embodiments, the compositions are substantially purified (with respect to non-RPE cells) preparations comprising differentiated RPE cells produced by the methods disclosed herein.

Compositions are also provided that include a scaffold, such as a polymeric carrier and/or an extracellular matrix, and an effective amount of the RPE cells produced by the methods disclosed herein. For example, the cells are provided as a monolayer of cells. The matrix material is generally physiologically acceptable and suitable for use in in vivo applications. For example, the physiologically acceptable materials include, but are not limited to, solid matrix materials that are absorbable and/or non-absorbable, such as small intestine submucosa (SIS), crosslinked or non-crosslinked alginate, hydrocolloid, foams, collagen gel, collagen sponge, polyglycolic acid (PGA) mesh, fleeces and bioadhesives.

Suitable polymeric carriers include porous meshes or sponges formed of synthetic or natural polymers, as well as polymer solutions. For example, the matrix is a polymeric mesh or sponge, or a polymeric hydrogel. Natural polymers that can be used include proteins such as collagen, albumin, and fibrin; and polysaccharides such as alginate and polymers of hyaluronic acid. Synthetic polymers include both biodegradable and non-biodegradable polymers. For example, biodegradable polymers include polymers of hydroxy acids such as polylactic acid (PLA), polyglycolic acid (PGA) and polylactic acid-glycolic acid (PGLA), polyorthoesters, polyanhydrides, polyphosphazenes, and combinations thereof. Non-biodegradable polymers include polyacrylates, polymethacrylates, ethylene vinyl acetate, and polyvinyl alcohols.

Polymers that can form ionic or covalently crosslinked hydrogels which are malleable can be used. A hydrogel is a substance formed when an organic polymer (natural or synthetic) is cross-linked via covalent, ionic, or hydrogen bonds to create a three-dimensional open-lattice structure which entraps water molecules to form a gel. Examples of materials which can be used to form a hydrogel include polysaccharides such as alginate, polyphosphazines, and polyacrylates, which are crosslinked ionically, or block copolymers such as PLURONICS™ or TETRONICS™ polyethylene oxide-polypropylene glycol block copolymers which are crosslinked by temperature or H, respectively. Other materials include proteins such as fibrin, polymers such as polyvinylpyrrolidone, hyaluronic acid and collagen.

In some embodiments, provided herein are compositions comprising a PLGA scaffold and an effective amount of the RPE cells obtained by the methods disclosed herein.

In some embodiments, provided pharmaceutical compositions can be packaged in a suitable container with written instructions for a desired purpose, such as the reconstitution of RPE cell function to improve a disease or abnormality of the retinal tissue. In some embodiments, the RPE cells produced by the disclosed methods may be engineered to form RPE, which can be used to replace degenerated RPE of a subject in need therein.

### Use of Retinal Pigment Epithelial Cells

Also provided herein are methods to produce an RPE or RPE-enriched cell population which can be used for a number of important research, development, and commercial purposes.

In some embodiments, the methods disclosed herein result in a cell population of at least or about 10⁶, 10⁷, 10⁸, 5×10⁸, 10⁹, 10¹⁰ cells (or any range derivable therein) comprising at least or about 90% (for example, at least or about 90%, 95%, 96%, 97%, 98%, 99%, 99.5%, or any range derivable therein) RPE cells.

In some embodiments, starting cells for the present methods may comprise the use of at least or about 10⁴, 10⁵, 10⁶, 10⁷, 10⁸, 10⁹, 10¹⁰, 10¹¹, 10¹², 10¹³ cells or any range derivable therein.

In some embodiments, the starting cell population may have a seeding density of at least or about 10¹, 10², 10³, 10⁴, 10⁵, 10⁶, 10⁷, 10⁸ cells/mL, or any range derivable therein.

The RPE cells produced by the methods disclosed herein may be used in any methods and applications currently known in the art for RPE cells.

### Compound Assessment and Screening

In some embodiments, provided herein is a method of assessing a compound, comprising assaying a pharmacological or toxicological property of the compound on the RPE cell. Also provided is a method of assessing a compound for an effect on a RPE cell, comprising: a) contacting the RPE cells provided herein with the compound; and b) assaying an effect of the compound on the RPE cells.

In some embodiments, RPE cells can be used commercially to screen for factors (such as solvents, small molecule drugs, peptides, oligonucleotides) or environmental conditions (such as culture conditions or manipulation) that affect the characteristics of such cells and their various progeny. For example, test compounds may be chemical compounds, small molecules, polypeptides, growth factors, cytokines, or other biological agents.

In some embodiments, a method includes contacting a RPE cell with a test agent and determining if the test agent modulates activity or function of RPE cells within the population. In some embodiments, screening assays are used for the identification of agents that modulate RPE cell proliferation or alter RPE cell differentiation. Screening assays may be performed in vitro or in vivo. Methods of screening and identifying ocular agents or RPE agents include those suitable for high-throughput screening. For example, RPE cells can be positioned or placed on a culture dish, flask, roller bottle or plate (e.g., a single multi-well dish or dish such as 8, 16, 32, 64, 96, 384 and 1536 multi-well plate or dish), optionally at defined locations, for identification of potentially therapeutic molecules. Libraries that can be screened include, for example, small molecule libraries, siRNA libraries, and adenoviral transfection vector libraries.

Other screening applications relate to the testing of pharmaceutical compounds for their effect on retinal tissue maintenance or repair. Screening may be done either because the compound is designed to have a pharmacological effect on the cells, or because a compound designed to have effects elsewhere may have unintended side effects on cells of this tissue type.

### Therapy and Transplantation

Other embodiments can also provide use of RPE cells to enhance ocular tissue maintenance and repair for any condition in need thereof, including retinal degeneration or significant injury.

To determine suitability of cell compositions for therapeutics administration, the cells can first be tested in a suitable animal model. In one aspect, the RPE cells are evaluated for their ability to survive and maintain their phenotype in vivo. Cell compositions are administered to immunodeficient animals (e.g., nude mice or animals rendered immunodeficient chemically or by irradiation). Tissues are harvested after a period of growth, and assessed as to whether the pluripotent stem cell-derived cells are still present.

A number of animals are available for testing of the suitability of the RPE cell compositions. For example, the Royal College of Surgeon's (RCS) rat is a well-known model of retinal dystrophy (Lund et al., 2006). In addition, RPE cell suitability and survival can be determined by transplantation (e.g., subcutaneous or subretinal) in matrigel in immunodeficient animals such as NOG mice (Kanemura et al., 2014).

The human RPE cells described herein, or a pharmaceutical composition including these cells, can be used for the manufacture of a medicament to treat a condition in a patient in need thereof. The RPE cells can be previously cryopreserved. In some embodiments, the disclosed RPE cells are derived from iPSCs, and thus can be used to provide “personalized medicine” for patients with eye diseases. In some embodiments, somatic cells obtained from patients can be genetically engineered to correct the disease-causing mutation, differentiated into RPE, and engineered to form an RPE tissue. This RPE tissue can be used to replace the endogenous degenerated RPE of the same patient. Alternatively, iPSCs generated from a healthy donor or from HLA homozygous “super-donors” can be used. RPE cells can be treated in vitro with certain factors, such as pigment epithelium-derived factor (PEDF), transforming growth factor (TGF)-beta, and/or retinoic acid to generate an anti-inflammatory and immunosuppressive environment in vivo.

Various eye conditions may be treated or prevented by the introduction of the RPE cells obtained using the methods disclosed herein. The conditions include retinal diseases or disorders generally associated with retinal dysfunction or degradation, retinal injury, and/or loss of retinal pigment epithelium. Conditions that can be treated include, without limitation, degenerative diseases of the retina, such as Stargardt's macular dystrophy, retinitis pigmentosa, macular degeneration (such as age-related macular degeneration), glaucoma, and diabetic retinopathy. Additional conditions include Lebers congenital amaurosis, hereditary or acquired macular degeneration, Best disease, retinal detachment, gyrate atrophy, choroideremia, pattern dystrophy, other dystrophies of the RPE, and RPE and retinal damage due to damage caused by any one of photic, laser, inflammatory, infectious, radiation, neovascular or traumatic injury. In certain embodiments, methods are provided for treating or preventing a condition characterized by retinal degeneration, comprising administering to a subject in need thereof an effective amount of a composition comprising RPE cells. These methods can include selecting a subject with one or more of these conditions, and administering a therapeutically effective amount of the RPE cells sufficient to treat the condition and/or ameliorate symptoms of the condition. The RPE cells may be transplanted in various formats. For example, the RPE cells may be introduced into the target site in the form of cell suspension, or adhered onto a matrix, extracellular matrix or substrate such as a biodegradable polymer, as a monolayer, or a combination. The RPE cells may also be transplanted together (co-transplantation) with other retinal cells, such as with photoreceptors. In some embodiments, the RPE cells are produced from iPSCs from the subject to be treated, and thus are autologous. In other embodiments, the RPE cells are produced from an MHC-matched donor.

In some embodiments, the RPE cells can be used for autologous RPE grafts to those subjects suitable for receiving regenerative medicine. The RPE cells may be transplanted in combination with other retinal cells, such as with photoreceptors. Transplantation of the RPE cells produced by the disclosed methods can be performed by various techniques and methods known in the art, e.g., those described in U.S. Pat. Nos. 5,962,027 and 6,045,791. In some embodiments, the transplantation is performed via pars pana vitrectomy surgery followed by delivery of the cells through a small retinal opening into the sub-retinal space or by direct injection. In some embodiments, the RPE cells can be introduced into the target site in the form of cell suspension, adhered onto a matrix, such as extracellular matrix, or provided on substrate such as a biodegradable polymer. In some embodiments, the RPE cells can also be transplanted together (co-transplantation) with other cells, such as retinal cells with photoreceptors. Accordingly, a composition comprising RPE cells obtained by the methods disclosed herein is provided. In some embodiments, these RPE cells include a tyrosinase enhancer operably linked to a promoter and a nucleic acid encoding a marker. In other embodiments, the RPE cells also include a second constitutive promoter operably linked to a nucleic acid encoding a second marker.

### Kits

In some embodiments, a kit that includes, for example, one or more media and components for the production of RPE cells is provided. The reagent system may be packaged either in aqueous media or in lyophilized form, where appropriate. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed. However, various combinations of components may be comprised in a vial. The components of the kit may be provided as dried powder(s). When reagents and/or components are provided as a dry powder, the powder can be reconstituted by the addition of a suitable solvent. It is envisioned that the solvent may also be provided in another container means. The kits also will typically include a means for containing the kit component(s) in close confinement for commercial sale. Such containers may include injection or blow molded plastic containers into which the desired vials are retained. The kit can also include instructions for use, such as in printed or electronic format, such as digital format.

### EXAMPLES

### Example 1. Preparation of Starting Population of iPSCs

A starting population of iPSCs can be reprogrammed from somatic cells by any methods known in the art. This Example describes an exemplary method.

Blood is collected from the donor by peripheral blood draw, and mononuclear cells (MNCs) are collected. Isolated peripheral blood mononuclear cells (PBMCs) are cryopreserved and stored in liquid nitrogen until use.

CD34+ or CD71+ cells are purified from PBMCs, e.g., using magnetic beads and expanded. Cells are then reprogrammed by introducing the reprogramming vector. In some embodiments, cells are reprogrammed using the CTS™ CytoTune™-iPS 2.1 Sendai Reprogramming Kit, following the manufacturer's protocol, to yield iPSCs cultured on vitronectin-coated plates. Post introduction of the reprogramming vector, e.g., transduction of the Sendai reprogramming viruses, cells are monitored for emergence of cell clumps/colonies indicative of reprogrammed cells. These colonies are subsequently picked, and optionally expanded for one passage, to yield a starting population of iPSCs for the next steps.

### Example 2. Preparation of Starting Population of iPSCs

This Example describes a further exemplary method to produce a starting population of iPSCs.

Blood is collected from the donor by peripheral blood draw, and mononuclear cells (MNCs) are collected. Isolated peripheral blood mononuclear cells (PBMCs) are cryopreserved and stored in liquid nitrogen until use.

CD34+ or CD71+ cells are purified from PBMCs, e.g., using magnetic beads and expanded. Cells are then reprogrammed by introducing the reprogramming vector. In some embodiments, cells are reprogrammed using the CTS™ CytoTune™-iPS 2.1 Sendai Reprogramming Kit, following the manufacturer's protocol, to yield iPSCs cultured on vitronectin-coated plates. Post introduction of the reprogramming vector, e.g., transduction of the Sendai reprogramming viruses, cells are monitored for emergence of cell clumps/colonies indicative of reprogrammed cells. A pooled culture of iPSCs were thereby obtained, optionally passaged for one or more times, and optionally cryopreserved, to yield a starting population of iPSCs for the next steps.

### Example 3. Generation of iPSCs Essentially Free of Exogeneous Viral Residuals

A starting population of iPSCs was obtained as described in Example 1 or Example 2, where Sendai virus reprogramming vector was used. The iPSCs were dissociated into a single cell suspension. Cells were counted, diluted as appropriate, and seeded at a density of about 5,000 to 10,000 cells/well in a 6-well plate coated with vitronectin in ESSENTIAL 8 (E8) medium and cultured at 37° C. The medium was changed daily or every other day with fresh E8 medium until individual iPSC colonies emerge. The medium was supplemented with RevitaCell Supplement, containing a ROCK inhibitor, to increase iPSC survival after dissociation into single cells while the cells were not adhered to the plate.

The well(s) with cells having the lowest degree of differentiation was selected. Cells of the selected well were dissociated into a single cell suspension. Cells were reseeded at a density of about 1,500 to 3,000 cells/well in a 6-well plate coated with vitronectin in E8 medium and cultured at 37° C. until individual iPSC colonies emerge. The medium was changed daily or every other day with fresh E8 medium. The medium was supplemented with RevitaCell Supplement to increase iPSC survival after dissociation into single cells while the cells were not adhered to the plate.

After 2 to 4 days culturing at 37° C. to allow individual iPSC colonies emerge, the culturing temperature was elevated to 38.5° C. for 5 to 6 days and back to 37° C. for another 1 to 2 days. The culturing temperature was kept constant at 37° C. for the control. The cells were not allowed to overgrow to prevent merging of individual colonies.

Subsequently, one iPSC colony per well was manually picked and passaged for 1 to 8 times. The resulting iPSCs were subjected to immunostaining and RT-qPCR analyses of Sendai viral residuals. The immunostaining assay used a fluorescently-labeled rabbit polyclonal anti-Sendai virus antibody. The TaqMan kit (Thermo Fisher, containing a labeled TaqMan probe and Sendai virus cDNA template) was used for the RT-qPCR.

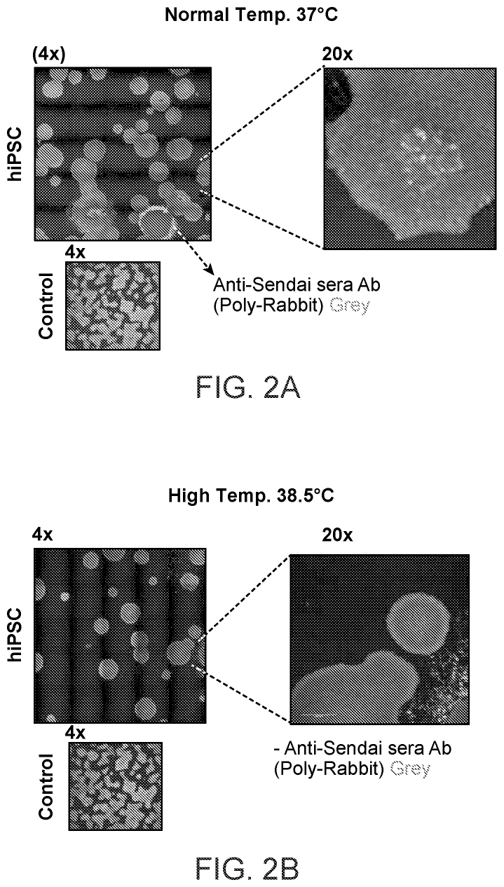

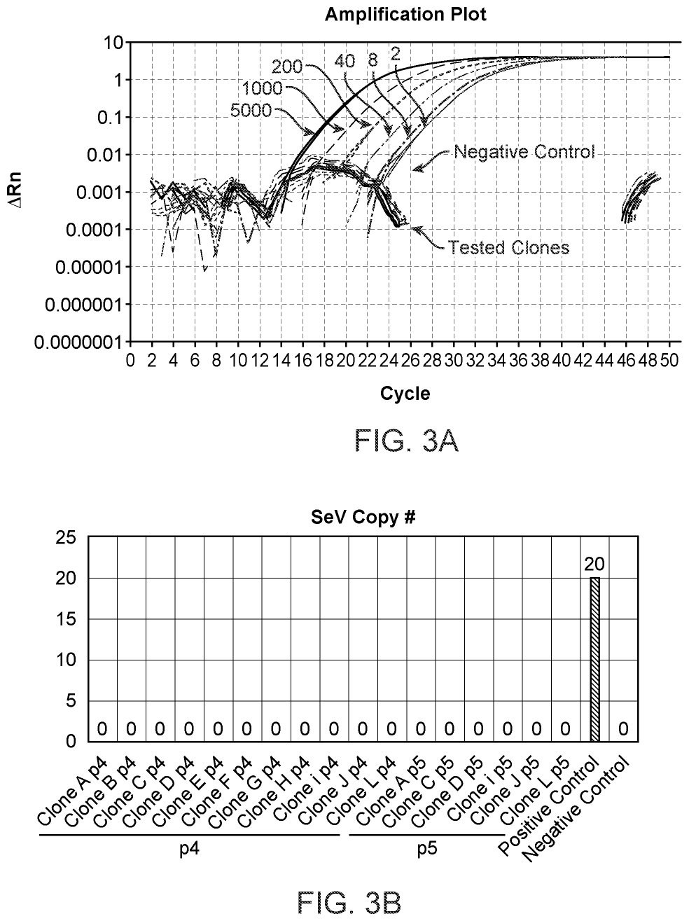

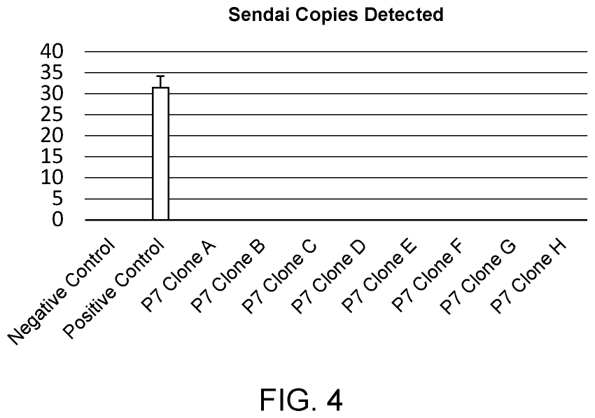

FIGS. 2B, 3A and 3B show that iPSCs generated from starting iPSC population obtained as described in Example 1 and colonies treated with elevated temperature were clear of Sendai virus. FIG. 4 shows that iPSCs generated from starting iPSC population obtained as described in Example 2 and colonies treated with elevated temperature were clear of Sendai virus. The data demonstrate introducing temperature elevation early at early stage prior to clone selection can generate vector-free iPSCs at early passages (less than 8).

### Example 4. Generation of RPE Cells

### Example 4.1. Preparation of Single Cell iPSC

A population of iPSCs essentially free of exogeneous viral residuals is obtained as described in Example 2. The iPSCs are dissociated into a single cell suspension in E8™ medium to remove any aggregates or embryoid bodies. Cells are counted, diluted as appropriate (e.g., to about 1×10⁵ cells/mL in room temperature E8 medium), and seeded at a density of about 200,000 cells/well in a 6-well plate coated with vitronectin and placed in a humidified incubator at 37° C. After about 18-24 hours, the medium is aspirated and fresh E8 medium is added to the culture. The cells are cultured in the E8 medium for about 2 days after seeding for proper adherence to the plate. Blebbistatin (e.g., 2.5 μM) is added to the E8 medium to increase iPSC survival after dissociation into single cells while the cells are not adhered to a culture vessel.

### Example 4.2. Differentiation of iPSCs into RPE cells

Subsequently, the single cell iPSCs are cultured in various differentiation media for deriving RPE cells.

On day 3, the E8™ medium is aspirated and a retinal induction medium (RIM) (e.g., the RIM in Table 1) at room temperature is added. The cells are cultured in the RIM for about two to four days. Each day, the media is aspirated and fresh RIM is added.

The cells are next cultured in a retinal differentiation medium (RDM) (e.g., the RDM in Table 1) for about seven to fourteen days. Each day, the media is aspirated and room temperature RDM is added to the cells to produce differentiated retinal cells.

Subsequently, the cells are cultured in a retinal medium (RM) (e.g., the RM in Table 1) for seven to ten days to derive RPE cells. The medium is changed daily with room temperature RM, resulting in RPE cells.

For maturation of the RPE cells, the cells are cultured in an RPE maturation medium (RPE-MM) (e.g., the RPE-MM in Table 1) for five to ten days. The medium is changed every other day with room temperature RPE-MM. Then, the cells are dissociated in a cell dissociation enzyme and reseeded on vitronectin coated plates. At this stage, the derived PRE cells can be cryopreserved, e.g., in xeno-free CS10 medium. Alternatively, to continue RPE maturation, plated cells are cultured for another approximately fifteen days.

### Example 4.3. Continued Maturation of RPE Cells

For continued maturation of the RPE cells, the cells are dissociated in a cell dissociation enzyme (e.g., TRYPLE™) and reseeded on a degradable scaffold assembly in a specialized SNAPWELL™ design for 1-2 weeks in the RPE-MM with a MEK inhibitor (e.g., PD325901). This resulted in differentiated, polarized, and confluent monolayers of functional RPE cells, which can be cryopreserved at this stage in xeno-free CS10 medium.

The mature RPE cells can be further developed into functional RPE cell monolayers that function as an intact RPE tissue by continued culture in the RPE-MM with additional small molecules such as primary cilium inducers like PGE2 or aphidicolin. Without being bound by theory, these primary cilium inducers suppress the canonical WNT pathway, induce cell cycle exit in the cells, and induce apical-basal polarization in the RPE monolayer. RPE maturity can alternatively be induced by canonical WNT pathway inhibitors such as rWP2 and endo-rWRI that also induce cell cycle exit in RPE cells to promote RPE maturation. The cells are cultured in this medium for another 2-3 weeks to obtain mature and functional RPE cell monolayers.

### Example 4.4. Depletion of Contaminating Cells

RPE cells obtained as described in Example 3 may have residual contaminating non-RPE cells as well as immature RPE cells (collectively referred to as the “contaminating cells”), both of which can be separated and removed to yield a mature RPE-enriched cell population. The contaminating cells can be removed from the culture by any methods known in the art, for example, magnetic activated cell sorting (MACS®), fluorescent activated cell sorting (FACS), or single cell sorting.

In some embodiments, the MACS® methodology, which separate various cell populations depending on their surface antigens, is used to separate the contaminating cells from the mature RPE-enriched cells. CD24, CD56, and/or CD90 are used as surface markers to separate/remove the contaminating cells. These markers are expressed on PSCs but lost during differentiation of stem cells to RPE cells.

In some embodiments, the inclusion of MEK inhibitor PD0325901 at a concentration of 1 μM in media for certain windows of time beginning on Day 2 post iPSC plating through the end of the differentiation process, including culture post depletion of contaminating cells, may improve both purity of the RPE population (meaning a decrease in contaminating cells) and maturity of the resulting RPE population. Inclusion of 1M PD0325901 has been shown to improve both purity and maturity of the RPE population when included in RDM as well as in RPE-MM (approximately Days 42 through 50) of the RPE process described herein.

In some embodiments, a decrease in the percentage of fetal bovine serum from 5 percent to 0.5-1 percent in RPE-MM and in medium used during the depletion of contaminating cells may improve both purity of the RPE population and maturity of the resulting RPE population.

### Example 4.5. Characterization of RPE Cells

Characterization of the RPE cells is performed with various methods known in the art, such as flow cytometry, immunostaining, and electrophysiological techniques, for their purify (e.g., RPE-specific markers), maturity, and functionality (e.g., transepithelial electrical potential/resistance measurement).

TABLE 1
Exemplary medium components

| | | | |
|---|---|---|---|
| Component | Vendor | Cat# | Final Conc. |

Essential 8 Medium

| | | | |
|---|---|---|---|
| Essential 8 ™ Basal Medium | Life Technologies | A1517001 | 98% |
| Essential 8 ™ Supplement | Life Technologies | | 2% |

Essential 8 Plating Medium

| | | | |
|---|---|---|---|
| Complete Essential 8 ™ Medium | Life Technologies | As prepared above | 100% |
| Essential 8 ™ Supplement | Life Technologies | | 2% |
| Blebbistatin | Sigma | B0560 | 2.5 |

Retinal Induction Medium (RIM)

| DMEM/F12 | Life Technologies | 11330-032 | 99% | |
|---|---|---|---|---|
| CTS ™ KnockOut ™ SR | Life Technologies | A1099201 | 1.5% | |
| XenoFree | | | | |
| MEM non-essential AA | Life Technologies | 11140 | 0.1 | mM |
| Sodium Pyruvate | Life Technologies | 11360-070 | 1 | mM |
| CTS ™ N-2 Supplement | Life Technologies | A13707-01 | 1% | |
| B-27 ® Supplement (+VitA) | Life Technologies | 17504-044 | 2% | |
| Ascorbic Acid | Sigma | A4544 | 200 | μM |
| LDN-193189 | Stemgent | 04-0074 | 10 | nM |
| SB 431542 | R&D Systems | 1614/10 | 1.0 | μM |
| CKI-7 Dihydrochloride | Sigma | C0742 | 0.5 | μM |
| AF-IGF-1 | R&D Systems | AFL291 | 1 | ng/ml |

Retinal Differentiation Medium (RDM)

| DMEM/F12 | Life Technologies | 11330-032 | 99% | |
|---|---|---|---|---|
| CTS ™ KnockOut ™ SR | Life Technologies | A1099201 | 1.5% | |
| XenoFree | | | | |
| MEM non-essential AA | Life Technologies | 11140 | 0.1 | mM |
| Sodium Pyruvate | Life Technologies | 11360-070 | 1 | mM |
| N-2 Supplement | Life Technologies | A13707-01 | 1% | |
| B-27 ® Supplement (+VitA) | Life Technologies | 17504-044 | 2% | |
| Ascorbic Acid | Sigma | A4544 | 200 | μM |
| LDN-193189 | Stemgent | 04-0074 | 100 | nM |
| SB 431542 | R&D Systems | 1614/10 | 10 | μM |
| CKI-7 Dihydrochloride | Sigma | C0742 | 5 | μM |
| AF-IGF-1 | R&D Systems | AFL291 | 10 | nM |
| PD0325901 | Stemgent | 04-0006 | 1 | μM |

Retinal Medium (RM) + Nicotinamide and Activin

| DMEM/F12 | Life Technologies | 11330-032 | 99% | |
|---|---|---|---|---|
| CTS ™ KnockOut ™ SR | Life Technologies | A1099201 | 1.5% | |
| XenoFree Kit | | | | |
| MEM non-essential AA | Life Technologies | 11140 | 0.1 | mM |
| Sodium Pyruvate | Life Technologies | 11360-070 | 1 | mM |
| CTS ™ N-2 Supplement | Life Technologies | A13707-01 | 1% | |
| B-27 ® Supplement (+VitA) | Life Technologies | 17504-044 | 2% | |
| Ascorbic Acid | Sigma | A4544 | 200 | μM |
| Nicotinamide | Sigma | N0636 | 10 | mM |
| Activin A | R&D Systems | 338-AC | 100 | ng/mL |

RPE-Maturation Medium (RPE-MM)

| MEM Alpha | Life Technologies | 12571-063 | 99% | |
|---|---|---|---|---|
| Fetal Bovine Serum | Hyclone | SH30071.03 | 5% | |
| CTS ™ N-2 Supplement | Life Technologies | A13707-01 | 1% | |
| MEM non-essential AA | Life Technologies | 11140 | 0.1 | mM |
| Sodium Pyruvate | Life Technologies | 11360-070 | 1 | mM |
| Taurine | Sigma | T4571 | 250 | μg/mL |
| Hydrocortisone | Sigma | H6909 | 20 | μg/mL |
| 3,3′,5-Triiodo-L-thyronine | Sigma | T-5516 | 0.013 | μg/L |

RPE-MM + PD0325901

| MEM Alpha | Life Technologies | 12571-063 | 99% | |
|---|---|---|---|---|
| Fetal Bovine Serum | Hyclone | SH30071.03 | 5% | |
| CTS ™ N-2 Supplement | Life Technologies | A13707-01 | 1% | |
| MEM non-essential AA | Life Technologies | 11140 | 0.1 | mM |
| Sodium Pyruvate | Life Technologies | 11360-070 | 1 | mM |
| Taurine | Sigma | T4571 | 250 | μg/mL |
| Hydrocortisone | Sigma | H6909 | 20 | μg/mL |
| 3,3′,5-Triiodo-L-thyronine | Sigma | T-5516 | 0.013 | μg/L |
| PD0325901 | Stemgent | 04-0006 | 1 | μM |

RPE-MM + PD0325901

| MEM Alpha | Life Technologies | 12571-063 | 99% | |
|---|---|---|---|---|
| Fetal Bovine Serum | Hyclone | SH30071.03 | 5% | |
| CTS ™ N-2 Supplement | Life Technologies | A13707-01 | 1% | |
| MEM non-essential AA | Life Technologies | 11140 | 0.1 | mM |
| Sodium Pyruvate | Life Technologies | 11360-070 | 1 | mM |
| Taurine | Sigma | T4571 | 250 | μg/mL |
| Hydrocortisone | Sigma | H6909 | 20 | μg/mL |
| 3,3′,5-Triiodo-L-thyronine | Sigma | T-5516 | 0.013 | μg/L |
| PGE2 | R&D Systems | 2296 | 50 | μM |

### Equivalents/Other Embodiments

Those skilled in the art will recognize or be able to ascertain using no more than routine experimentation, many equivalents to the specific embodiments described herein. Such equivalents are intended to be encompassed by the following claims.

What is claimed is:

1. A method of producing a clonal population of induced pluripotent stem cells (iPSCs) that is essentially free of exogeneous vector residuals, the method comprising, in the following order:
a) obtaining a starting population of iPSCs produced using a reprogramming vector;
b) seeding the iPSCs at a low density and culturing in a culture medium;
c) optionally repeating step b) for one or more times;
d) optionally culturing the iPSCs at an elevated temperature;
e) selecting a single iPSC colony and passaging for 1-8 times to produce the population of iPSCs that is essentially free of exogeneous vector residuals.

2. A method of manufacturing induced pluripotent stem cells (iPSCs) essentially free of exogeneous vector residuals, comprising, in the following order:
a) obtaining a starting population of iPSCs produced using a reprogramming vector;
b) seeding the iPSCs at a low density and culturing in a culture medium;
c) optionally repeating step b) for one or more times;
d) optionally culturing the iPSCs at an elevated temperature;
e) selecting a single iPSC colony and passaging for 1-8 times to yield the iPSCs essentially free of exogeneous vector residuals.

3. The method of claim 1 or 2, wherein the starting population of iPSCs is produced from somatic cells of a human individual.

4. The method of claim 3, wherein the somatic cells are blood cells.

5. The method of claim 4, wherein the blood cells are peripheral blood cells.

6. The method of claim 4 or 5, wherein the blood cells are CD34+ or CD71+ enriched cells.

7. The method of any one of claims 1-6, wherein the starting population of iPSCs is a polyclonal pool of iPSCs.

8. The method of any one of claims 1-7, wherein step a) comprises
i) generating a polyclonal pool of iPSCs produced using a reprogramming vector;
ii) optionally passaging the iPSCs one or more times; and
iii) optionally cryopreserving iPSCs resulting from step i) or step ii), thereby giving the starting population of iPSCs.

9. The method of any one of claims 1-8, wherein the starting population of iPSCs has not been passaged or has been passaged once prior to its use in the method.

10. The method of any one of claims 1-9, wherein the reprograming vector is a non-integrating viral vector.

11. The method of claim 10, wherein the viral vector is a Sendai viral vector, wherein the Sendai viral vector comprises one or more temperature-sensitive mutations.

12. The method of any one of claims 1-11, wherein step b) is repeated for one time.

13. The method of any one of claims 1-11, wherein step b) is repeated for two times.

14. The method of any one of claims 1-13, wherein step b), each time performed, independently comprises dissociating the iPSCs into essentially single cells prior to seeding.

15. The method of any one of claims 1-14, wherein step b), each time performed, independently comprises seeding the iPSCs at a density of about 1 to about 1500 cells/cm².

16. The method of claim 15, the iPSCs are seeded, each time performed, independently at a density of about 140 to about 350 cells/cm² or about 470 to about 1150 cells/cm².

17. The method of any one of claims 1-16, wherein step b), when performed the last time, comprises seeding the iPSCs at a clonal density.

18. The method of claim 17, wherein the clonal density is about 140 to about 350 cells/cm².

19. The method of claim 17, wherein the clonal density is about 340 cells/cm².

20. The method of any one of claims 1-19, wherein step b), each time performed, independently comprises seeding the iPSCs in the culture medium supplemented with a Rho-associated protein kinase (ROCK) inhibitor.

21. The method of any one of claims 1-20, wherein the culture medium of step b) is a fully defined medium.

22. The method of claim 21, wherein the culture medium is Essential 8 medium.

23. The method of any one of claims 1-22, wherein step b), each time performed, independently comprises culturing the iPSCs at about 37.0° C. to about 39.0° C.

24. The method of any one of claims 1-23, wherein step b), each time performed, independently comprises seeding the iPSCs at a density of about 140 to about 350 cells/cm² or about 470 to about 1150 cells/cm² in the culture medium supplemented with a ROCK inhibitor and subsequently culturing in the culture medium at about 37.0° C. in 5% CO₂ incubation atmosphere until single iPSC colonies emerge.

25. The method of any one of claims 1-24, wherein step b), when performed the last time, comprises seeding the iPSCs at a density of about 140 to about 350 cells/cm² in the culture medium supplemented with a ROCK Inhibitor and subsequently culturing the iPSCs in the culture medium at about 37.0° C. in 5% CO₂ incubation atmosphere for 1-3 days.

26. The method of any one of claims 1-25, wherein step d) is not performed.

27. The method of any one of claims 1-25, wherein step d) is performed.

28. The method of claim 27, wherein step d) comprises culturing the iPSCs in Essential 8 medium in 5% CO₂ incubation atmosphere.

29. The method of claim 27 or 28, wherein the elevated temperature is about 38.0° C. to about 39.0° C.

30. The method of any one of claims 27-29, wherein step d) comprises culturing the iPSCs at the elevated temperature for 5-8 days.

31. The method of any one of claims 27-30, wherein step d) further comprises culturing the iPSCs at about 37.0° C. for at least 1 day.

32. The method of any one of claims 27-31, wherein step d) comprises culturing the iPSCs in Essential 8 medium at about 38.0° C. to about 39.0° C. in 5% CO₂ incubation atmosphere for about 6 days and subsequently at about 37.0° C. in 5% CO₂ incubation atmosphere for 1-2 days.

33. The method of any one of claims 1-32, wherein the essentially free of exogeneous viral residuals is determined by quantitative real-time polymerase chain reaction (qRT-PCR) or quantitative polymerase chain reaction (qPCR).

34. A method of producing a clonal population of induced pluripotent stem cells (iPSCs) that is essentially free of exogeneous viral residuals, the method comprising, in the following order:
a) obtaining a starting population of iPSCs produced using a Sendai viral vector containing temperature-sensitive mutations,
b1) dissociating the iPSCs into essentially single cells and seeding at a density of about 470 to about 1150 cells/cm² in a culture medium supplemented with a ROCK inhibitor and subsequently culturing in the culture medium at about 37.0° C. in 5% CO₂ incubation atmosphere until single iPSC colonies emerge;
b2) dissociating the iPSCs into essentially single cells and seeding at a density of about 140 to about 350 cells/cm² in the culture medium supplemented with a ROCK inhibitor and subsequently culturing in the culture medium at about 37° C. in 5% CO₂ incubation atmosphere for about 3 days;
c) culturing the iPSCs in the culture medium at about 38.0° C. to about 39.0° C. in 5% CO₂ incubation atmosphere for about 6 days and subsequently at about 37.0° C. in 5% CO₂ incubation atmosphere for 1-2 days; and
d) selecting a single iPSC colony and culturing for less than 8 passages to produce the population of iPSCs that is essentially free of exogeneous viral residuals.

35. A method of manufacturing induced pluripotent stem cells (iPSCs) essentially free of exogeneous viral residuals, comprising, in the following order:
a) obtaining a starting population of iPSCs produced using a Sendai viral vector containing temperature-sensitive mutations,
b1) dissociating the iPSCs into essentially single cells and seeding at a density of about 470 to about 1150 cells/cm² in a culture medium supplemented with a ROCK inhibitor and subsequently culturing in the culture medium at about 37.0° C. in 5% CO₂ incubation atmosphere until single iPSC colonies emerge;
b2) dissociating the iPSCs into essentially single cells and seeding at a density of about 140 to about 350 cells/cm² in the culture medium supplemented with a ROCK inhibitor and subsequently culturing in the culture medium at about 37.0° C. in 5% CO₂ incubation atmosphere for about 3 days;
c) culturing the iPSCs at in the culture medium at about 38.0° C. to about 39.0° C. in 5% CO₂ incubation atmosphere for about 6 days and subsequently at about 37.0° C. in 5% CO₂ incubation atmosphere for 1-2 days; and
d) selecting a single iPSC colony and culturing for less than 8 passages to yield iPSCs essentially free of exogeneous viral residuals.

36. A method of producing human retinal pigment epithelial (RPE) cells, comprising, in the following order:
a) obtaining a population of iPSCs that is essentially free of exogeneous viral residuals produced according to any one of claims 1 and 3-34;
b) seeding the iPSCs and culturing in a retinal induction medium to initiate differentiation of the cells into retinal lineage cells;
c) culturing the retinal lineage cells in a retinal differentiation medium to further differentiate the retinal lineage cells;
d) culturing the cells in retinal medium to form differentiating RPE cells; and
e) culturing the differentiating RPE cells in a RPE maturation medium, thereby producing human RPE cells.

37. A method of manufacturing human retinal pigment epithelial (RPE) cells, comprising:
a) obtaining a population of iPSCs essentially free of exogeneous viral residuals according to any one of claims 1-35;
b) seeding the iPSCs and culturing in a retinal induction medium to initiate differentiation of the cells into retinal lineage cells;
c) culturing the retinal lineage cells in a retinal differentiation medium to further differentiate the retinal lineage cells;
d) culturing the cells in retinal medium to form differentiating RPE cells; and
e) culturing the differentiating RPE cells in a RPE maturation medium, thereby yielding human RPE cells.

38. The method of claim 36 or 37, wherein the method does not comprise the formation of embryoid bodies.

39. The method of any one of claims 36-38, wherein the iPSCs of step a) have been dissociated into single cells.

40. The method of any one of claims 36-38, wherein step b) comprises dissociating the iPSCs into essentially single cells prior to seeding.

41. The method of any one of claims 36-40, wherein step b) comprises seeding the iPSCs
i) at a cell density of about 5000 to about 40000 cells/cm²,
ii) without a feeder layer,
iii) in a fully defined culture medium, and/or
iv) in a xeno-free culture medium.

42. The method of any one of claims 36-41, wherein b) comprises culturing iPSCs on a matrix.

43. The method of claim 42, wherein the matrix comprises at least one recombinant cellular adhesion protein.

44. The method of claim 43, wherein the at least one cellular adhesion protein is laminin, vitronectin, or fibronectin.

45. The method of claim 43 or 44, wherein the cellular adhesion protein is human protein.

46. The method of any one of claims 36-45, wherein the retinal induction medium comprises a WVNT pathway inhibitor, a TGFβ pathway inhibitor, a BMP pathway inhibitor, and insulin growth factor 1 (IGF1).

47. The method of any one of claims 36-46, wherein the retinal differentiation medium comprises a WVNT pathway inhibitor, a TGFβ pathway inhibitor, a BMP pathway inhibitor, a MEK inhibitor, and IGF1.

48. The method of any one of claims 36-47, wherein step e) comprises dissociating the differentiating RPE cells, reseeding the RPE cells, and culturing the RPE cells in the RPE maturation medium, wherein the RPE maturation medium comprises a MEK inhibitor.

49. The method of claim 48, wherein the RPE cells are reseeded on a degradable scaffold in the RPE maturation medium.

50. The method of any one of claims 36-49, wherein the RPE maturation medium comprises at least one primary cilium inducer.

51. The method of claim 50, wherein the primary cilium inducer is prostaglandin E2 (PGE2) or aphidicolin.

52. The method of any one of claims 36-51, further comprising cryopreserving the human RPE cells.

53. A method for producing human retinal pigment epithelial (RPE) cells, comprising, in the following order:
a) obtaining a population of iPSCs that is essentially free of exogeneous viral residuals produced according to any one of claims 1 and 3-34, and dissociating the iPSCs into essentially single cells in a fully defined medium;
b) seeding the iPSCs and culturing on laminin, vitronectin, or a combination thereof, in a retinal induction medium comprising LDN193189, CKI-7, and SB431542 to initiate differentiation of the cells into retinal lineage cells;
c) culturing the retinal lineage cells in a retinal differentiation medium comprising LDN193189, CKI-7, SB431542, and PD0325901 to further differentiate the retinal lineage cells;
d) culturing the cells in retinal medium comprising nicotinamide and Activin A to form differentiating RPE cells; and
e) culturing the differentiating RPE cells in a RPE maturation medium, thereby producing human RPE cells;
wherein the method does not comprise the formation of embryoid bodies.

54. A method for manufacturing human retinal pigment epithelial (RPE) cells, comprising:
a) obtaining iPSCs essentially free of exogeneous viral residuals according to any one of claims 1-35, and dissociating the iPSCs into essentially single cells in a fully defined medium;
b) seeding the iPSCs and culturing on laminin, vitronectin, or a combination thereof, in a retinal induction medium comprising LDN193189, CKI-7, and SB431542 to initiate differentiation of the cells into retinal lineage cells;
c) culturing the retinal lineage cells in a retinal differentiation medium comprising LDN193189, CKI-7, SB431542, and PD0325901 to further differentiate the retinal lineage cells;
d) culturing the cells in retinal medium comprising nicotinamide and Activin A to form differentiating RPE cells; and
e) culturing the differentiating RPE cells in a RPE maturation medium, thereby yielding human RPE cells;
wherein the method does not comprise the formation of embryoid bodies.

55. A pharmaceutical composition comprising human RPE cells produced or manufactured according to any one of claims 36-54, a pharmaceutically acceptable carrier, and optionally a scaffold.

56. The pharmaceutical composition of claim 55, wherein the scaffold is a poly(lactic-co-glycolic acid) (PLGA) scaffold.
